SEQUENCE LISTING (10 sequences)

SEQ ID NO: 1 - DNA, 2249 bp, Homo sapiens
  CDS (503)..(1330)
gggcagtagaggggtacgcgccttacagatcctttcctctgttaagtgtaagggatgggagtaaagagctaaggaagaatgcctcccaccagcatctgacacattaaaaaaattaccgtgaggacagaggttctttagagtcactcttcgctaacatttattttcatttgttttccacaagctgaatcatgaccctatagccccccactgtacaccaaggtcgtattgattgaattgttacaaacaagtctgcagggaagcctctctgggagatgttgtgtttctggtggacaccaacatcaacccccaacatgcacgcagtgtgcggaacttcttgtacattctggtaaacagcttcaacgtcagcagtgagaccatccgtgtgggtctggccaagtatagcgatgtgccccattcagagttcttgctttccacctaccaccgcaaaggtgatgtgttgagacacattcggcagtttcaatttaagcctgggggcaagaagatgggcctggccctgaagttcattctagaccaccacttccaggaagcatcagggagccgggcaagccaagaggtacctcagatcgccgtggtgatcagcagtgggccagttgaggaccatgtccatggacctgcgaaggcattgaggaaggcaggcatcctgctttatgctattggcgtcagagatgcagtttgggcagagctcagggaaattgccagtagtcctcaggagaattttacctcctttgttcctaacttctctggtctgagcaatctcgcccagaagctgcggcaggaactctgtgatacgttggcaaaggcagctccacatgttgaccacgtctctccagcttgcagagaagcagccctggcagacattgtgtttctagtagacagttcaactagcatcggaccccaaaacttccagaaagtcaagaacttcctttactccgtcatcttggggcttgacatcagcagtgaccgtgtccgagtgggacttgcccagtataatgacaacatctacccagccttccagctgaaccagcaccctctgaagagcatgatcttggagcagatccagaatctgccataccgcacaggaggcacaaacacagggagcgccctggagtttatcaggaccaactatttgactgaggagtctggcagtcgggccaaggacagagttcctcagatagttatcctggtgactgacggggagtcaaatgatgaggtccaggaggtggctgaccggttgaaagaagatggagttgttgtgttatgtggtaggggtcaatgtccaggatgtccaagagttgcaaaaaataaccagtgagccatttgagaagtttctcttcaacactgaaaacttcaatatcctgcaggatttttcaggaagtattcttcagactctgtgctcagcagtggagggtaagataaaagcgtgcgcaactgctgtcccggctgacttaatattcctcattgaggaatttagcagggttaggcaacccaatttccaacaagttgtcaatttcctaaagaccattgtcagttctctaaacattcgtcctgatactgtgagacttggcttggtcttctacagtgaggaaccacgacttgaattttcactggatgcatttcagaatccagccaaaatcttggagcatttggacaaattaacctaccgggaaagaaaaggaaggacgaagactggtgctgcattagatttcctgaggaacgaggttttcattcaggagaagggtagccggtccaaccacggtgtgcagcagatagctgtagtcatcacggaaggcttctcccaagacagagtatctagacccgcttcccacctccgaagggcaggcgtcaccatctatgcagtgggcacccacaatgtctcagagagtaaggacctggagaagatagcatcatatcctccttggaagaattcagtccccctggaatcctttttgcagctctccatagtaggaagcaagcttacacaccagctctgctctgagatggtggacagtaaagtttccattagtgggacgagctatcccctacaagaaggttgtgtgcacatcgaaaaggcagatatttacttccttattgatgggtctggcagcatctatccagaagattttcttgagatgaaggtgttcatgaatgaagtgataaagatgttccagatcgggcccaacagagtacagtttggaatcattcagtactcagacaaaattaaaagtaaa

SEQ ID NO: 2 - protein, 276 aa, Homo sapiens
MGLALKFILDHHFQEASGSRASQEVPQIAVVISSGPVEDHVHGPAKALRKAGILLYAIGVRDAVWAELREIASSPQENFTSFVPNFSGLSNLAQKLRQELCDTLAKAAPHVDHVSPACREAALADIVFLVDSSTSIGPQNFQKVKNFLYSVILGLDISSDRVRVGLAQYNDNIYPAFQLNQHPLKSMILEQIQNLPYRTGGTNTGSALEFIRTNYLTEESGSRAKDRVPQIVILVTDGESNDEVQEVADRLKEDGVVVLCGRGQCPGCPRVAKNNQ

SEQ ID NO: 3 - DNA, 28 bp, Artificial Sequence
  Description of Artificial Sequence Synthetic primer
ctcactgctgacgttgaagctgtttacc

SEQ ID NO: 4 - DNA, 27 bp, Artificial Sequence
  Description of Artificial Sequence Synthetic primer
gtgtccaccagaaacacaacatctccc

SEQ ID NO: 5 - DNA, 28 bp, Artificial Sequence
  Description of Artificial Sequence Synthetic primer
tgggcagagctcagggaaattgccagta

SEQ ID NO: 6 - DNA, 28 bp, Artificial Sequence
  Description of Artificial Sequence Synthetic primer
agaagctgcggcaggaactctgtgatac

SEQ ID NO: 7 - DNA, 20 bp, Artificial Sequence
  Description of Artificial Sequence Synthetic primer
gcggcaggaactctgtgata

SEQ ID NO: 8 - DNA, 20 bp, Artificial Sequence
  Description of Artificial Sequence Synthetic primer
atgtcaagccccaagatgac

SEQ ID NO: 9 - DNA, 21 bp, Artificial Sequence
  Description of Artificial Sequence Synthetic oligonucleotide
guagacaguucaacuagcatt

SEQ ID NO: 10 - DNA, 21 bp, Artificial Sequence
  Description of Artificial Sequence Synthetic oligonucleotide
ugcuaguugaacugucuactt

(19) United States
(12) Patent Application Publication
(10) Pub. No.: US 2011/0065102 A1
(43) Pub. Date: Mar. 17, 2011

(54) OSTEOARTHRITIS-SENSITIVE GENE

(75) Inventors: Shiro Ikegawa, Tokyo (JP); Yoshihiro Miyamoto, Tokyo (JP)

(73) Assignee: RIKEN, Saitama (JP)

(21) Appl. No.: 12/670,662

(22) PCT Filed: Jul. 25, 2008
(86) PCT No.: PCT/JP2008/063399

§ 371 Date: Oct. 20, 2010

(30) Foreign Application Priority Data
Jul. 27, 2007 (JP) 2007-196017

(51) Int. Cl. C12Q 1/68; C12Q 1/02; C07H 21/02; C07K 14/47; C07H 21/04

(52) U.S. Cl. 435/6; 435/29; 536/24.5; 530/350; 536/23.5

(57) ABSTRACT

It is an object of the present invention to provide a method and kit for diagnosing osteoarthritis which involves genetic diagnosis or hemodiagnosis. The present invention provides a method for diagnosing a genetic susceptibility of a subject to osteoarthritis, which comprises detecting at least one polymorphism selected from polymorphisms existing in a gene (Dual Intracellular on Willebrand factor A gene; DIVA gene), encoding the amino acid sequence of SEQ ID NO: 2 or an amino acid sequence substantially homologous thereto, in a DNA-containing sample collected from the subject, wherein an allele frequency of one of alleles is higher in arbitrary osteoarthritis group than in arbitrary non-osteoarthritis group.

### TECHNICAL FIELD

The present invention relates to a gene associated with osteoarthritis and the identification of polymorphisms in the gene associated with the aforementioned disease, and the prevention and/or treatment of osteoarthritis based on such gene and polymorphisms.

### BACKGROUND ART

Osteoarthritis is a common disease that causes the pain of joint (particularly, knee joint and hip joint) and a limited range of motion. Approximately 6% of adults 30 years old and above frequently suffer from gonalgia. The gonalgia is diagnosed as osteoarthritis by X-ray examination. Osteoarthritis frequently leads to not only disability, but also financial problems, in elderly people. Managing osteoarthritis is an urgent need worldwide.

Known risk factors of osteoarthritis include age, sex, family history, past history of joint injuries, occupational factors and obesity. Genetic predisposition is also evident, as firstly described by Kellgren et al. in knee osteoarthritis (Non-Patent Document 1). A few susceptibility genes of osteoarthritis, such as ASPN (Non-Patent Document 2), FRZB (Non-Patent Document 3) and GDF5 (Non-Patent Document 4) were identified and confirmed in multiple populations to date.

Genomewide association study is a powerful approach for complex human disease such as osteoarthritis. Susceptibility genes of several diseases were identified using Genomewide association study (Non-Patent Documents 5-7).
Non-Patent Document 1: Kellgren, J. H., J. S. Lawrence, et al. (1963). “Genetic Factors in Generalized Osteo-Arthrosis.” Ann Rheum Dis 22: 237-55.
Non-Patent Document 2: Kizawa, H., I. Kou, et al. (2005). “An aspartic acid repeat polymorphism in asporin inhibits chondrogenesis and increases susceptibility to osteoarthritis.” Nat Genet 37(2): 138-44.
Non-Patent Document 3: Loughlin, J., B. Dowling, et al. (2004). “Functional variants within the secreted frizzled-related protein 3 gene are associated with hip osteoarthritis in females.” Proc Natl Acad Sci U.S.A. 101(26): 9757-62.
Non-Patent Document 4: Miyamoto, Y., A. Mabuchi, et al. (2007). “A functional polymorphism in the 5′ UTR of GDF5 is associated with susceptibility to osteoarthritis.” Nat Genet 39(4): 529-33.
Non-Patent Document 5: Ozaki, K., Y. Ohnishi, et al. (2002). “Functional SNPs in the lymphotoxin-alpha gene that are associated with susceptibility to myocardial infarction.” Nat Genet 32(4): 650-4.
Non-Patent Document 6: Klein, R. J., C. Zeiss, et al. (2005). “Complement factor H polymorphism in age-related macular degeneration.” Science 308(5720): 385-9.
Non-Patent Document 7: Kubo, M., J. Hata, et al. (2007). “A nonsynonymous SNP in PRKCH (protein kinase C eta) increases the risk of cerebral infarction.” Nat Genet.

### DISCLOSURE OF THE INVENTION

### Problems to be Solved by the Invention

At present, as a method for diagnosing osteoarthritis, there is applied only diagnostic imaging such as X-ray examination or MRI (magnetic resonance imaging). However, such diagnostic imaging has been problematic in that it is often ineffective before the onset of a disease or at the initial stage of a disease, and in that it is less objective. Thus, it is an object of the present invention to provide a method for diagnosing osteoarthritis which involves genetic diagnosis or hemodiagnosis. It is another object of the present invention to provide an agent for preventing and/or treating osteoarthritis. It is a further object of the present invention to provide a method for screening for an agent for preventing and/or treating osteoarthritis.

### Means for Solving the Problems

The present inventors have conducted intensive studies directed towards achieving the aforementioned objects. The inventors have carried out genome-wide association study on knee osteoarthritis. As a result, they have succeeded in identifying a novel gene encoding the amino acid sequence of SEQ ID NO: 2 (Dual Intracellular on Willebrand factor A gene; DIVA gene), which has never been reported before. Moreover, the present inventors have found that the missense SNP of DIVA is significantly associated with knee osteoarthritis, and that the DIVA protein binds to tubulin but the osteoarthritis susceptibility allele of such missense SNP has a weak binding ability to tubulin. The present invention has been completed based on these findings.

The present invention provides the following.
(1) A method for diagnosing a genetic susceptibility of a subject to osteoarthritis, which comprises detecting at least one polymorphism selected from polymorphisms existing in a gene (Dual Intracellular Von Willebrand factor A gene; DIVA gene), encoding the amino acid sequence of SEQ ID NO: 2 or an amino acid sequence substantially homologous thereto (Dual Intracellular on Willebrand factor A gene; DIVA gene), in a DNA-containing sample collected from the subject, wherein an allele frequency of one of alleles is higher in arbitrary osteoarthritis group than in arbitrary non-osteoarthritis group.
(2) The method according to (1), wherein the polymorphism is selected from the group consisting of the polymorphisms of registration numbers rs9864422, rs7639618, and rs11718863 in the NCBI SNP Database, and polymorphisms that are in a linkage disequilibrium state with the polymorphisms, having a linkage disequilibrium coefficient D′ of 0.9 or greater.
(3) The method according to (2), wherein the subject is determined to have high genetic susceptibility to osteoarthritis when a genotype of registration number rs11718863 in the NCBI SNP Database is T and a genotype of registration number rs7639618 is G.
(4) The method according to any one of (1) to (3), wherein the osteoarthritis is knee osteoarthritis.
(5) The method according to any one of (1) to (4), wherein presence or absence of a genetic polymorphism is detected, the genetic polymorphism causing an amino acid at position 169 in the amino acid sequence of SEQ ID NO: 2 to alter from Asn to an amino acid other than Asn.
(6) The method according to any one of (1) to (5), wherein presence or absence of a genetic polymorphism is detected, the genetic polymorphism causing an amino acid at position 260 in the amino acid sequence of SEQ ID NO: 2 to alter from Tyr to an amino acid other than Tyr.
(7) A method for screening an agent for preventing and/or treating osteoarthritis, which comprises administering a test substance to cells that express a gene encoding a protein consisting of the amino acid sequence of SEQ ID NO: 2 or an amino acid sequence substantially homologous thereto, and selecting a substance that inhibits expression or function of the protein.
(8) An agent for preventing and/or treating osteoarthritis, which comprises an agent for inhibiting expression or function of a protein consisting of the amino acid sequence of SEQ ID NO: 2 or an amino acid sequence substantially homologous thereto.
(9) A protein consisting of the following amino acid sequence (a) or (b):
(a) the amino acid sequence of SEQ ID NO: 2; or
(b) an amino acid sequence, which comprises a deletion, substitution, insertion, and/or addition of one or more amino acid residues with respect to the amino acid sequence of SEQ ID NO: 2, and which is able to bind to tubulin.
(10) DNA encoding the protein of (9).
(11) DNA consisting of any one of the following nucleotide sequences (a) to (c): (a) the nucleotide sequence of SEQ ID NO: 1;
(b) a nucleotide sequence, which comprises a deletion, substitution, insertion, and/or addition of one or more nucleotides with respect to the nucleotide sequence of SEQ ID NO: 1, and which encodes an amino acid sequence that is able to bind to tubulin; and
(c) a nucleotide sequence, which hybridizes with the nucleotide sequence of SEQ ID NO: I or a sequence complementary thereto under stringent conditions, and which encodes an amino acid sequence that is able to bind to tubulin.

### Effects of the Invention

According to the present invention, it becomes possible to diagnose the disease susceptibility of a subject to osteoarthritis by examining the disease susceptibility polymorphisms of the DIVA gene (by genetic diagnosis, hemodiagnosis, etc.). That is to say, it becomes possible to carry out the preclinical diagnosis, risk diagnosis, and early diagnosis of osteoarthritis. Moreover, according to the present invention, it becomes also possible to prevent and/or treat osteoarthritis and to carry out a causal treatment on osteoarthritis by controlling the expression of the DIVA gene and the physiological activity thereof. Furthermore, according to the present invention, it becomes also possible to develop an agent for preventing and/or treating osteoarthritis by screening for an agent for controlling the expression of the DIVA gene.

### BEST MODE FOR CARRYING OUT THE INVENTION

The present invention will be described more in detail below.

The method for diagnosing genetic susceptibility to osteoarthritis according to the present invention is characterized in that it comprises detecting at least one polymorphism selected from the group consisting of polymorphisms existing in a gene encoding the amino acid sequence of SEQ ID NO: 2 or an amino acid sequence substantially homologous thereto (Dual Intracellular Von Willebrand factor A gene; DIVA gene) in a DNA-containing sample collected from a subject, in which an allele frequency of one of the two alleles is higher in arbitrary osteoarthritis group than in arbitrary non-osteoarthritis group. Specifically, the method for diagnosing genetic susceptibility to osteoarthritis of the present invention is characterized in that it comprises detecting a polymorphism in the DIVA gene of a subject.

The term “(genetic) polymorphism” is used in the present specification to mean an alteration (substitution, deletion, insertion, dislocation, inversion, etc.) of one or more nucleotides on genomic DNA, wherein such alteration exists at a frequency of 1% or more in a population. Examples of such polymorphism include: the substitution of one nucleotide with another nucleotide (SNP); deletion or insertion of one to several tens of nucleotides (DIP); and a portion in which a sequence (a unit) consisting of two to several tens of nucleotides repeatedly exists, wherein the repeat count is different (a polymorphism whose repeating unit is 2 to 4 nucleotides is referred to as a microsatellite polymorphism, and a polymorphism whose repeating unit is several to several tens of nucleotides is referred to as a variable number of tandem repeat (VNTR)). The polymorphism that can be used in the diagnostic method of the present invention may be any one of the above-described types, as long as it satisfies the following conditions. It is preferably SNP.

The polymorphism that can be used in the diagnostic method of the present invention is not particularly limited, as long as it satisfies the following conditions (1) and (2):
(1) it is a polymorphism existing in the DIVA gene; and
(2) an allele frequency of one of the two alleles is significantly higher in arbitrary osteoarthritis group than in arbitrary non-osteoarthritis group.

Examples of a polymorphism existing in the DIVA gene include known polymorphisms registered at the NCBI SNP Database (http://www.ncbi.nim.nih.SNP/), the JSNP Database (http://snp.ims.u-tokyo.ac.jp/), the Applied Biosystems Homepage (http://www.appliedbiosystems.com/index.cfm), and the like. Specific examples of such polymorphism existing in the DIVA gene include the polymorphisms with registration numbers rs9864422, rs7639618, and rs11718863 in the NCBI SNP Database, and polymorphisms that are in a linkage disequilibrium state with the aforementioned polymorphisms, having a linkage disequilibrium coefficient D′ of 0.9 or greater.

Preferred examples of a polymorphism that can be used in the diagnostic method of the present invention include the polymorphisms with registration numbers rs9864422, rs7639618, and rs11718863 in the NCBI SNP Database, and polymorphisms that are in a linkage disequilibrium state with the aforementioned polymorphisms, having a linkage disequilibrium coefficient D′ of 0.9 or greater. Herein, the “linkage disequilibrium coefficient D′” can be obtained by the formula as described below, wherein each allele of the first SNP of two SNPs is represented by (A, a), each allele of the second SNP is represented by (B, b), and the frequencies of four haplotypes (AB, Ab, aB, and ab) are represented by P_{AB}, P_{Ab}, P_{aB}, and P_{ab}, respectively.

D′=(P_{AB}P_{ab}−P_{Ab}P_{aB})/Min[(P_{AB}+Pₐₛ)(P_{aB}+P_{ab}), (P_{AB}+P_{Ab})(P_{Ab}+P_{ab})]

[wherein Min[(P_{AB}P_{aB})(P_{aB}+P_{ab}), (P_{AB}+P_{Ab})(P_{Ab}+P_{ab})] means that (P_{AB}+P_{aB}+P_{ab}) or (P_{AB}+P_{Ab})(P_{Ab}+P_{ab}), which is a smaller value, is adopted.]

A polymorphism having D′ of preferably 0.95 or greater, more preferably 0.99 or greater, and most preferably 1, is used in the present invention.

Among the aforementioned polymorphisms, those in which an allele frequency of one of the two alleles is significantly higher in arbitrary osteoarthritis group than in arbitrary non-osteoarthritis group can be used in the diagnostic method of the present invention. Hereinafter, in the present specification, such polymorphism may be referred to as an “osteoarthritis marker polymorphism” at times.

The osteoarthritis group and the non-osteoarthritis group are not particularly limited in terms of their size (the number of samples), the background of each sample (for example, origin, age, sex, disease, etc.), and the like, as long as they are groups consisting of samples that are sufficient for obtaining statistically reliable results. An example of osteoarthritis is knee osteoarthritis. In addition, since informed consent must be ethically obtained from sample donors, in general, a group of patients suffering from diseases other than osteoarthritis in a certain medical institution, or a group of subjects who were diagnosed not to have osteoarthritis by group medical examination in a certain region, is preferably used as a non-osteoarthritis group.

In the present invention, polymorphism(s) selected from the group consisting of the polymorphisms with registration numbers rs9864422, rs7639618, and rs11718863 in the NCBI SNP Database, and polymorphisms that are in a linkage disequilibrium state with the aforementioned polymorphisms, having a linkage disequilibrium coefficient D′ of 0.9 or greater, can be used in the diagnosis. Polymorphisms detected by the diagnostic method of the present invention may be either one, or two or more of the aforementioned polymorphisms.

Any of known SNP detection methods can be used in the detection of polymorphisms in the diagnostic method of the present invention. Classical detection methods include: a method, which comprises carrying out hybridization while precisely controlling stringency, for example, according to the method of Wallace et al. (Proc. Natl. Acad. Sci. U.S.A., 80, 278-282 (1983)), for example, using, as a sample, genomic DNA extracted from the cells of a subject and the like, and also using, as a probe, a nucleic acid comprising a nucleotide sequence consisting of approximately 15 to 500 contiguous nucleotides containing nucleotides at a polymorphic site, having registration number rs9864422, rs7639618, and rs11718863 in the NCBI SNP Database, so as to detect only a sequence completely complementary to the probe; and a method, which comprises carrying out hybridization, while gradually decreasing a denaturation temperature, using a mix probe in which either one of the aforementioned nucleic acid and a nucleic acid whose nucleotides at the polymorphic site of the aforementioned nucleic acid are substituted with other nucleotides is labeled, and the other one is unlabeled, so that a sequence completely complementary to one probe is allowed to previously hybridize, thereby preventing a cross-reaction with a probe having mismatch. Herein, as a labeling agent, a radioisotope, an enzyme, a fluorescent substance, a luminescent substance, or the like can be used. Specific examples of such radioisotope used herein include [¹²⁵I], [¹³¹I], [³H], and [¹⁴C]. As an enzyme used herein, a stable enzyme having large specific activity is preferable. Specific examples of such enzyme used herein include β-galactosidase, β-glucosidase, alkaline phosphatase, peroxidase, and malate dehydrogenase. Specific examples of a fluorescent substance used herein include fluorescamine and fluorescein isothiocyanate. Specific examples of a luminescent substance used herein include luminol, a luminol derivative, luciferin, and lucigenin.

Preferably, a polymorphism can be detected by various methods described in WO 03/023063, such as an RFLP method, a PCR-SSCP method, ASO hybridization, a direct sequencing method, an ARMS method, a denaturing gradient gel electrophoresis method, an RNase A cleavage method, a chemical cleavage method, a DOL method, a TaqMan PCR method, an invader method, a MALDI-TOF/MS method, a TDI method, a molecular beacon method, a dynamic allele-specific hybridization method, a padlock probe method, a UCAN method, a nucleic acid hybridization method using a DNA chip or a DNA microarray, and an ECA method (see page 17, line 5 to page 28, line 20, WO 03/023063). Hereinafter, representative polymorphism detection methods, the TaqMan PCR method and the invader method, will be described more in detail.

### (a) TaqMan PCR Method

TaqMan PCR method is a method that involves PCR using a fluorescently labeled allele-specific oligonucleotide (a TaqMan probe) and Taq DNA polymerase. As such TaqMan probe, there is used an oligonucleotide consisting of a nucleotide sequence of approximately 15-30 contiguous nucleotides containing nucleotides at a polymorphic site with registration number rs9864422, rs7639618 or rs11718863 in the NCBI SNP Database. The 5-terminus of this probe is labeled with a fluorescent dye such as FAM or VIC, and the 3-terminus thereof is labeled with a quencher (a quenching substance) such as TAMRA. Since such quencher absorbs fluorescence energy in this state, no fluorescence is detected. Thus, it is preferable that probes be prepared for both alleles, and that the prepared probes be then labeled with fluorescent dyes having different fluorescence wavelengths (for example, either one allele is labeled with FAM, and the other allele is labeled with VIC) for one-time detection. In addition, in order to prevent the occurrence of a PCR elongation reaction from the TaqMan probe, the 3′-terminus thereof is phosphorylated. When PCR is carried out using Taq DNA polymerase and primers designed such that they can amplify a partial sequence of genomic DNA containing a region to be hybridizing with the TaqMan probe, the TaqMan probe hybridizes with template DNA, and at the same time, an elongation reaction occurs from the PCR primers. Thereafter, when such elongation reaction progresses, the hybridized TaqMan probe is cleaved by the 5′ nuclease activity of the Taq DNA polymerase, and the fluorescent dye is released, so that the influence of the quencher disappears, and fluorescence is thereby detected. As a result of amplification of the template, fluorescence intensity is exponentially increased.

### (b) Invader Method

Differing from the TaqMan PCR method, in invader method, an allele-specific oligonucleotide (an allele probe) is not labeled. A sequence (flap) that is not complementary to template DNA exists on the 5′-terminal side of a polymorphic site, and a complementary sequence specific to the template exists on the 3′-terminal side thereof. In the invader method, there are further used: an oligonucleotide having a complementary sequence specific to the 3′-terminal side of the polymorphic site of the template (invader probe; nucleotides corresponding to the polymorphic site at the 5′-terminus of this probe are arbitrarily determined); and an FRET (fluorescence resonance energy transfer) probe characterized in that it has a sequence capable of adopting a hairpin structure on the 5′-terminal side, and in that a sequence that ranges from a nucleotide making a pair with the nucleotide at the 5′-terminus when such hairpin structure is formed, to nucleotides on the 3′-terminal side, is complementary to the flap of the allele probe. The 5′-terminus of the FRET probe is fluorescently labeled (for example, with FAM or VIC), and a quencher (for example, TAMRA) binds to a portion therearound. Thus, no fluorescence is detected in this state (hairpin structure).

If the allele probe and the invader probe are allowed to react with genomic DNA used as a template, the 3′-terminus of the invader probe invades the polymorphic site, when the three components complementarily bind to one another. If the single-stranded portion of the allele probe (that is, a flap portion on the 5′-terminal side from the nucleotides at the polymorphic site) is cleaved with an enzyme (cleavase) recognizing the structure of the polymorphic site, the flap complementarily binds to the FRET probe, and the polymorphic site of the flap invades the hairpin structure of the FRET probe. As a result that cleavase recognizes this structure and cleaves it, the fluorescent dye that labels the terminus of the FRET probe is released, so that the influence of the quencher disappears and fluorescence is detected. An allele probe, in which the nucleotides at the polymorphic site do not match with the template, is not cleaved with cleavase. However, since such non-cleaved allele probe is able to hybridize with the FRET probe, fluorescence is detected in this case as well. However, because reaction efficiency is different, an allele probe, in which the nucleotides at the polymorphic site match with the template, has fluorescence intensity significantly stronger than that of the allele probe, in which the above nucleotides do not match with the template.

In general, before template DNA is allowed to react with 3 types of probes and cleavase, it is preferable that the template DNA has previously been amplified by PCR using primers capable of amplifying regions containing portions to be hybridizing with the allele probe and the invader probe.

As a result of the aforementioned examination of polymorphisms, when arbitrary osteoarthritis group has an allele that is significantly higher than that of arbitrary non-osteoarthritis group, and particularly, when such allele is determined as a homozygote, the subjects can be diagnosed to have high genetic susceptibility to the osteoarthritis. For instance, the subject is determined to have high genetic susceptibility to osteoarthritis when a genotype of registration number rs11718863 in the NCBI SNP Database is T and a genotype of registration number rs7639618 is G.

The present invention is able to provide a kit used in the aforementioned diagnostic method of the present invention. That is to say, the diagnostic kit of the present invention is characterized in that it comprises at least one pair of nucleic acid probes and/or primers that are able to detect one or more polymorphisms selected from the group consisting of polymorphisms existing in the DIVA gene, in which an allele frequency of one of the two alleles is higher in arbitrary osteoarthritis group than in arbitrary non-osteoarthritis group.

Specifically, the nucleic acid probe used in the diagnostic kit of the present invention is a nucleic acid hybridizing with genomic DNA in a region containing nucleotides at a polymorphic site to be detected by the aforementioned diagnostic method of the present invention, and the length of such nucleic acid (the base length of a portion hybridizing with the genomic DNA) is not particularly limited, as long as it is specific to a target portion and it can easily detect polymorphisms. The length of the nucleic acid is, for example, approximately 15 nucleotides or more, preferably approximately 15 to approximately 500 nucleotides, more preferably approximately 15 to approximately 200 nucleotides, and further preferably approximately 15 to approximately 50 nucleotides.

The probe may comprise an additive sequence suitable for the detection of polymorphisms (a sequence that is not complementary to genomic DNA). For instance, the allele probe used in the aforementioned invader method has an additive sequence called “flap” at the 5′-terminus of nucleotides at a polymorphic site.

Moreover, the probe may be labeled with a suitable labeling agent, such as a radioisotope (for example, ¹²⁵I, ¹³¹I, ³H, or ¹⁴C), an enzyme (for example, β-galactosidase, β-glucosidase, alkaline phosphatase, peroxidase, or malate dehydrogenase), a fluorescent substance (for example, fluorescamine or fluorescein isothiocyanate), or a luminescent substance (for example, luminol, a luminol derivative, luciferin, or lucigenin). Otherwise, a quencher (a quenching substance) for absorbing fluorescence energy generated from a fluorescent substance (for example, FAM or VIC) may also be allowed to bind to a region around the fluorescent substance. In such embodiment, a fluorescent substance is separated from a quencher during the detection reaction, so that fluorescence is detected.

Preferably, the nucleic acid probe used in the diagnostic kit of the present invention is a nucleic acid comprising a nucleotide sequence consisting of approximately 15 to approximately 500 nucleotides, preferably approximately 15 to approximately 200 nucleotides, and more preferably approximately 15 to approximately 50 nucleotides, which contains nucleotides at a polymorphic site selected from the group consisting of the polymorphisms with registration numbers rs9864422, rs7639618, and rs11718863 in the NCBI SNP Database, and polymorphisms that are in a linkage disequilibrium state with the aforementioned polymorphisms, having a linkage disequilibrium coefficient D′ of 0.9 or greater.

The nucleic acid primer used in the diagnostic kit of the present invention is not particularly limited, as long as it is designed to specifically amplify the region of genomic DNA containing nucleotides at a polymorphic site to be detected by the aforementioned diagnostic method of the present invention. The primer may comprise an additive sequence suitable for the detection of polymorphisms (a sequence that is not complementary to genomic DNA), such as a linker sequence.

Moreover, the primer may be labeled with a suitable labeling agent, such as a radioisotope (for example, ¹²⁵I, ¹³¹I, ³H, or ¹⁴C), an enzyme (for example, β-galactosidase, β-glucosidase, alkaline phosphatase, peroxidase, or malate dehydrogenase), a fluorescent substance (for example, fluorescamine or fluorescein isothiocyanate), or a luminescent substance (for example, luminol, a luminol derivative, luciferin, or lucigenin).

The nucleic acid probe or primer used in the diagnostic method of the present invention may be either DNA or RNA. In addition, it may be either a single strand or a double strand. In the case of a double strand, it may be any one of double-stranded DNA, double-stranded RNA, and a DNA/RNA hybrid. Accordingly, when a nucleic acid having a certain nucleotide sequence is described in the present specification, it should be understood that such nucleic acid is used to include all of a single-stranded nucleic acid having the certain nucleotide sequence, a single-stranded nucleic acid having a sequence complementary to the certain nucleotide sequence, and a hybrid thereof, unless otherwise specified.

The aforementioned nucleic acid probe or primer can be synthesized according to an ordinary method using a DNA/RNA automatic synthesizer, based on information regarding the nucleotide sequence with registration number rs9864422, rs7639618 or rs11718863 in the NCBI SNP Database, for example.

The aforementioned nucleic acid probe and/or primer can be dissolved separately (or in a mixed state, if possible) in water or in a suitable buffer (for example, a TE buffer) in an appropriate concentration (for example, 1 to 50 μM at a concentration of 2× to 20×), and they can be then preserved at approximately −20° C.

The diagnostic kit of the present invention may further comprise other ingredients necessary for the implementation of a polymorphism detection method, depending on the type of the method. When the present kit is used in the detection of polymorphisms by the TaqMan PCR method, for example, it may further comprise a 10×PCR reaction buffer, a 10×MgCl₂ aqueous solution, a 10×dNTPs aqueous solution, Taq DNA polymerase (5 U/μL), and the like.

The diagnostic kit of the present invention can be used in the diagnosis of genetic susceptibility to osteoarthritis, for example, to knee osteoarthritis.

The present invention also relates to the prevention and/or treatment of osteoarthritis by controlling (for example, by suppressing) the expression of DIVA and/or the activity thereof.

The description “an amino acid sequence substantially identical to the amino acid sequence of SEQ ID NO: 2” is used to mean an amino acid sequence, which has homology of approximately 50% or more, preferably approximately 60% or more, more preferably approximately 70% or more, further preferably approximately 80% or more, particularly preferably approximately 90% or more, and most preferably approximately 95% or more, with the amino acid sequence of SEQ ID NO: 2, and in which a protein having this amino acid sequence has activity substantially equivalent to that of a protein having the amino acid sequence of SEQ ID NO: 2. The term “homology” is used herein to mean the percentage (%) of amino acid residues identical or similar to all overlapping amino acid residues, in an optimal alignment obtained when two amino acid sequences are aligned based on a mathematical algorism known in the present technical field (wherein, in this algorism, introduction of a gap into either one or both of the two sequences can preferably be considered for such optimal alignment). The term “similar amino acid” is used to mean an amino acid similar to another amino acid in terms of physicochemical properties. Examples of such similar amino acid include amino acids classified into the same group, such as aromatic amino acids (Phe, Trp, and Tyr), aliphatic amino acids (Ala, Leu, Ile, and Val), polar amino acids (Gln and Asn), basic amino acids (Lys, Arg, and His), acidic amino acids (Glu and Asp), amino acids having a hydroxyl group (Ser and Thr), and amino acids having a small side chain (Gly, Ala, Ser, Thr, and Met). Substitution with such similar amino acids is predicted not to affect the phenotype of a protein (that is, it is a conservative amino acid substitution). Specific examples of such conservative amino acid substitution are publicly known in the present technical field, and are described in various publications (see Bowie et al., Science, 247: 1306-1310 (1990), for example).

Examples of an algorism for determining the homology of an amino acid sequence include: the algorism described in Karlin et al., Proc. Natl. Acad. Sci. U.S.A., 90: 5873-5877 (1993) [this algorism is incorporated into the NBLAST and)(BLAST programs (version 2.0) (Altschul et al., Nucleic Acids Res, 25: 3389-3402 (1997))]; the algorism described in Needlema et al., J. Mol. Biol., 48: 444-453 (1970) [this algorism is incorporated into GAP program in GCG software package]; the algorism described in Myers and Miller, CABIOS, 4: 11-17 (1988) [this algorism is incorporated into ALIGN program (version 2.0) as a part of CGC sequence alignment software package]; and the algorism described in Pearson et al., Proc. Natl. Acad. Sci. U.S.A., 85: 2444-2448 (1988) [this algorism is incorporated into FASTA program in GCG software package]. However, examples are not limited thereto.

The term “substantially equivalent” in the description “activity substantially equivalent to . . . ” is used to mean that the properties of two proteins are qualitatively (for example, physiologically or pharmacologically) equal.

Examples of the DIVA in the present invention include: (1) an amino acid sequence comprising a deletion of one or two or more (preferably approximately 1 to 30, more preferably approximately 1 to 10, and further preferably 1 to 5) amino acids from the amino acid sequence of SEQ ID NO: 2; (2) an amino acid sequence comprising an addition of one or two or more (preferably approximately 1 to 30, more preferably approximately 1 to 10, and further preferably 1 to 5) amino acids to the amino acid sequence of SEQ ID NO: 2; (3) an amino acid sequence comprising an insertion of one or two or more (preferably approximately 1 to 30, more preferably approximately 1 to 10, and further preferably 1 to 5) amino acids into the amino acid sequence of SEQ ID NO: 2; (4) an amino acid sequence comprising a substitution of one or two or more (preferably approximately 1 to 30, more preferably approximately 1 to 10, and further preferably 1 to 5) amino acids in the amino acid sequence of SEQ ID NO: 2 with other amino acids; and (5) a protein comprising an amino acid sequence formed by combining the aforementioned changes, which has activity substantially equivalent to a protein comprising the amino acid sequence of SEQ ID NO: 2. When an amino acid sequence comprises an insertion, deletion, or substitution, as described above, the position of such insertion, deletion, or substitution is not particularly limited, unless it impairs the activity of the protein.

The DIVA protein of the present invention can be produced by culturing a transformant prepared by the introduction of an expression vector containing a nucleic acid encoding DIVA so as to generate a DIVA protein, and then separating and/or purifying the DIVA protein from the obtained culture.

The type of a nucleic acid encoding DIVA is not particularly limited, as long as it comprises a nucleotide sequence encoding the aforementioned amino acid sequence of DIVA used in the present invention. The nucleic acid may be DNA, RNA, or a DNA/RNA chimera. It is preferably DNA.

DNA encoding DIVA may be: genomic DNA; cDNA from the cells [for example, liver cells, splenic cells, nerve cells, glia cells, pancreatic β cells, bone marrow cells, mesangium cells, Langerhans cells, epidermal cells, epithelial cells, goblet cells, endothelial cells, smooth muscle cells, fibroblasts, fibrocytes, myocytes, fat cells, immunocytes (for example, macrophages, T cells, B cells, natural killer cells, mast cells, neutrophils, basophils, eosinophils, or monocytes), megakaryocyte, synovial cells, chondrocytes, osteocytes, osteoblasts, osteoclasts, mammary glandular cells, liver cells or interstitial cells, or the precursor cells thereof, stem cells or cancer cells, etc.] of a human or other homeotherms (for example, a monkey, a bovine, a horse, a swine, a sheep, a goat, a rabbit, a mouse, a rat a guinea pig, a hamster, a chicken, etc.), or from all tissues or organs in which the aforementioned cells exist [for example, brain, individual sections in brain (for example, olfactory bulb, amygdaloid nucleus, basal ganglia, hippocampus, thalamus, hypothalamus, cerebral cortex, medulla oblongata, or cerebellum), spinal cord, pituitary gland, stomach, pancreas, kidney, liver, gonad, thyroid gland, gallbladder, bone marrow, adrenal gland, skin, muscle, lung, alimentary canal (for example, large intestine or small intestine), blood vessel, heart, thymus gland, spleen, submandibular gland, peripheral blood, prostate gland, testis, ovary, placenta, uterus, bone, joint, interspinal disk, adipose tissues (for example, brown adipose tissues or white adipose tissues), or skeletal muscle, etc.]; synthetic DNA; or the like. Also, such genomic DNA or cDNA encoding DIVA can be directly amplified by Polymerase Chain Reaction (hereinafter abbreviated as a “PCR method”) and Reverse Transcriptase-PCR (hereinafter abbreviated as an “RT-PCR method”) using a genomic DNA fraction and a total RNA or mRNA fraction as a template, respectively. Alternatively, such genomic DNA and cDNA encoding DIVA can also be cloned, respectively, from a genomic DNA library and cDNA library prepared by inserting each of a genomic DNA fragment and a total RNA or mRNA fragment prepared from the aforementioned cells and/or tissues into a suitable vector according to a colony or plaque hybridization method, a PCR method, or the like. The vector used for such library may be any one of a bacteriophage, a plasmid, a cosmid, and a phagemid.

An example of such DNA encoding DIVA is DNA encoding a protein comprising the amino acid sequence of SEQ ID NO: 2 or a protein having activity substantially equivalent thereto. Moreover, DNA that hybridizes with the aforementioned DNA under stringent conditions can also be used. For example, there can be used DNA comprising a nucleotide sequence having homology of approximately 60% or more, preferably approximately 70% or more, further preferably approximately 80% or more, and particularly preferably approximately 90% or more, with the nucleotide sequence of the aforementioned DNA.

Hybridization can be carried out in accordance with a known method or a method equivalent thereto, such as the method described in Molecular Cloning, 2^{nd} edition, (J. Sambrook et al., Cold Spring Harbor Lab. Press, 1989). In addition, when a commercially available library is used, hybridization can be carried out in accordance with the method described in an instruction manual included therewith. Hybridization' can be preferably carried out under stringent conditions.

Examples of such stringent conditions include conditions consisting of a sodium salt concentration of approximately 19 to approximately 40 mM, and preferably of approximately 19 to approximately 20 mM, and a temperature of approximately 50° C. to approximately 70° C., and preferably of approximately 60° C. to approximately 65° C. In particular, conditions consisting of a sodium salt concentration of approximately 19 mM and a temperature of approximately 65° C. are preferable. Persons skilled in the art can easily obtain desired stringency by altering, as appropriate, the salt concentration of a hybridization solution, a temperature applied during a hybridization reaction, a probe concentration, a probe length, the number of mismatches, a time required for a hybridization reaction, the salt concentration of a washing solution, a washing temperature, and the like.

DNA encoding DIVA can be cloned by amplifying it according to a PCR method using a synthetic DNA primer having a portion of a nucleotide sequence encoding the protein or peptide, or by hybridizing DNA incorporated into a suitable expression vector with a DNA fragment or synthetic DNA encoding a part of or the entire region of DIVA.

The nucleotide sequence of DNA can be converted according to a known method such as an ODA-LA PCR method, a Gapped duplex method or a Kunkel method, or a method equivalent thereto, using a known kit such as Mutan™-super Express Km (Takara Shuzo, Co., Ltd.) or Mutan™-K (Takara Shuzo, Co., Ltd.).

A nucleic acid comprising the nucleotide sequence encoding DIVA or a portion thereof (sense DIVA), or a nucleic acid comprising a nucleotide sequence complementary to the aforementioned nucleotide sequence or a portion thereof (antisense DIVA), is used as a probe or the like, so as to detect the abnormality of DNA or RNA (genetic abnormality) encoding DIVA in a human or other homeotherms (for example, a rat, a mouse, a hamster, a rabbit, a sheep, a goat, a swine, a bovine, a horse, a cat, a dog, a monkey, a chimpanzee, a bird, etc.). Thus, such nucleic acid is useful as an agent for genetic diagnosis, such as an agent for diagnosing the damage of the DNA, mutation, mRNA splicing abnormality, a decrease in the expression of such mRNA, amplification of the DNA, an increase in the expression of mRNA, etc. The type of a nucleic acid comprising a portion of the nucleotide sequence encoding DIVA is not particularly limited, as long as it has a length necessary as a probe (for example, approximately 15 nucleotides or more). Moreover, it is unnecessary for such nucleic acid to encode a partial peptide of DIVA.

The aforementioned genetic diagnosis, in which sense or antisense DIVA is used, can be carried out by a known method, such as Northern hybridization, quantitative RT-PCR, a PCR-SSCP method, allele-specific PCR, a PCR-SSOP method, a DGGE method, an RNase protection method, or a PCR-RFLP method.

A host is transformed with an expression vector containing the aforementioned DNA encoding DIVA, and the obtained transformant is then cultured, so as to produce the protein or peptide. Such expression vector containing the DNA encoding DIVA can be produced by cutting a DNA fragment of interest out of the DNA encoding DIVA, and then ligating the DNA fragment to a site downstream of a promoter in a suitable expression vector.

Examples of an expression vector used herein include: Escherichia coli-derived plasmids (for example, pBR322, pBR325, pUC12, and pUC13); Bacillus subtilis-derived plasmids (for example, pUB110, pTP5, and pC194); yeast-derived plasmids (for example, pSH19 and pSH15); bacteriophages such as λ phage; animal viruses such as retrovirus, vaccinia virus, and baculovirus; and pA1-11, pXT1, pRc/CMV, pRc/RSV and pcDNAI/Neo.

Any type of promoter may be used, as long as it is an appropriate promoter corresponding to a host used for the expression of a gene.

For example, when such host is an animal cell, an SRα promoter, an SV40 promoter, an LTR promoter, a CMV (cytomegalovirus) promoter, an HSV-TK promoter, or the like is used. Of these, a CMV promoter, an SRα promoter, and the like are preferable.

When the host is genus Escherichia, a trp promoter, a lac promoter, a recA promoter, a λ P_{L} promoter, an Ipp promoter, a T7 promoter, or the like is preferable.

When the host is genus Bacillus, an SP01 promoter, an SP02 promoter, a penP promoter, or the like is preferable.

When the host is yeast, a PH05 promoter, a PGK promoter, a GAP promoter, an ADH promoter, or the like is preferable.

When the host is an insect cell, a polyherin promoter, a P10 promoter, or the like is preferable.

As an expression vector, in addition to the aforementioned vectors, a vector comprising, as desired, an enhancer, a splicing signal, a poly(A) addition signal, a selection marker, an SV40 replication origin (hereinafter abbreviated as “SV40ori” at times), and the like may be used. Examples of a selection marker include: a dihydrofolate reductase (hereinafter abbreviated as “dhfr” at times) gene [methotrexate resistance]; an ampicillin resistance gene (hereinafter abbreviated as “Amp^{r}” at times); and a neomycin resistance gene (hereinafter abbreviated as “Neo^{r}” at times; G418 resistance). In particular, when dhfr gene-deficient Chinese hamster cells are used and a dhfr gene is used as a selection marker, a gene of interest can also be selected using a medium that does not contain thymidine.

Moreover, as necessary, a nucleotide sequence encoding a signal (or prepro) sequence corresponding to a host may be added to the 5′-terminal side of DNA encoding DIVA or a partial peptide thereof. When the host is genus Escherichia, there are used a PhoA/signal sequence, an OmpA/signal sequence, and the like. When the host is genus Bacillus, there are used an α-amylase/signal sequence, a subtilisin/signal sequence, and the like. When the host is a yeast, there are used an MFα/signal (prepro) sequence, an SUC2/signal sequence, and the like. When the host is an animal cell, there are used an insulin/signal (prepro) sequence, an α-interferon/signal sequence, an antibody molecule/signal sequence, and the like.

Examples of a host used herein include genus Escherichia, genus Bacillus, yeast, an insect cell, an insect, and an animal cell.

Examples of such genus Escherichia used herein include Escherichia coli K12/DH1 [Proceedings of the National Academy of Sciences of the U.S.A. (Proc. Natl. Acad. Sci. U.S.A.), Vol. 60, 160 (1968)], Escherichia coli JM103 [Nucleic Acids Research, Vol. 9, 309 (1981)], Escherichia coli JA221 [Journal of Molecular Biology, Vol. 120, 517 (1978)], Escherichia coli HB101 [Journal of Molecular Biology, Vol. 41, 459 (1969)], and Escherichia coli C600 [Genetics, Vol. 39, 440 (1954)].

Examples of such genus Bacillus used herein include Bacillus subtilis MI114 [Gene, Vol. 24, 255 (1983)] and Bacillus subtilis 207-21 [Journal of Biochemistry, Vol. 95, 87 (1984)].

Examples of such yeast used herein include Saccharomyces cerevisiae AH22, Saccharomyces cerevisiae AH22R⁻, Saccharomyces cerevisiae NA87-11A, Saccharomyces cerevisiae DKD-5D, Saccharomyces cerevisiae 20B-12, Schizosaccharomyces pombe NCYC1913, Schizosaccharomyces pombe NCYC2036, and Pichia pastoris KM71.

Examples of such insect cell used herein include: in a case in which the virus is AcNPV, Spodoptera frugiperda cells (Sf cells), MG1 cells from Trichoplusia ni midgut cells, High Five™ cells from TrichoPlusia ni egg cells, cells from Mamestra brassicae, and cells from Estigmena acrea; and in a case in which the virus is BmNPV, Bombyx mori N cells (BmN cells). Examples of the aforementioned Sf cells used herein include Sf9 cells (ATCC CRL1711) and Sf21 cells (Vaughn, J. L. et al., In Vivo, 13, 213-217 (1977)).

As an insect, silkworm and the like are used, for example [Maeda et al., Nature, Vol. 315, 592 (1985)].

Examples of an animal cell include monkey cell COS-7, Vero, Chinese hamster cell CHO (hereinafter abbreviated as a CHO cell), dhfr gene-deficient Chinese hamster cell CHO (hereinafter abbreviated as a CHO(dhfr⁻) cell), mouse L cell, mouse AtT-20, mouse myeloma cell, rat GH3, and human FL cell.

Transformation can be carried out in accordance with a known method, depending on the type of a host.

For example, genus Escherichia can be transformed by the method described in Proceedings of the National Academy of Sciences of the U.S.A. (Proc. Natl. Acad. Sci. U.S.A.), Vol. 69, 2110 (1972), Gene, Vol. 17, 107 (1982), or the like.

For example, genus Bacillus can be transformed by the method described in Molecular & General Genetics, Vol. 168, 111 (1979) or the like.

For example, yeast can be transformed by the method described in Methods in Enzymology, Vol. 194, 182-187 (1991), Proceedings of the National Academy of Sciences of the U.S.A. (Proc. Natl. Acad. Sci. U.S.A.), Vol. 75, 1929 (1978), or the like.

For example, an insect cell and an insect can be transformed by the method described in Bio/Technology, 6, 47-55 (1988) or the like.

For example, an animal cell can be transformed by the method described in Saibokogaku Bessatsu 8, Shin-Saibokogaku Jikken Protocol (Cell Technology, Suppl. 8, New Cell Technology Experimental Protocols), 263-267 (1995) (published by Shujunsha, Co., Ltd.), or Virology, Vol. 52, 456 (1973).

The transformant can be cultured according to a known method, depending on the type of a host.

For example, when a transformant whose host is genus Escherichia or genus Bacillus is cultured, a liquid medium is preferably used as a medium in the culture of the transformant. In addition, it is preferable for the medium to comprise a carbon source, a nitrogen source, an inorganic material, and the like, which are necessary for the growth of the transformant. Examples of a carbon source used herein include glucose, dextrin, soluble starch, and sucrose. Examples of a nitrogen source used herein include inorganic or organic substances such as ammonium salts, nitrates, corn steep liquor, peptone, casein, meat extract, soybean cake, and potato extract. Examples of an inorganic material used herein include calcium chloride, sodium dihydrogen phosphate, and magnesium chloride. Moreover, yeast extract, vitamins, a growth-promoting factor, and the like may also be added to the medium. The pH of the medium is preferably approximately pH 5 to 8.

When a transformant whose host is genus Escherichia is cultured, as a medium, an M9 medium containing glucose and casamino acid is preferably used [Miller, Journal of Experiments in Molecular Genetics, 431-433, Cold Spring Harbor Laboratory, New York 1972]. For efficient function of a promoter, as necessary, an agent such as 3β-indolylacrylic acid may be added to the medium, for example.

A transformant whose host is genus Escherichia is generally cultured at a temperature of approximately 15° C. to 43° C. for approximately 3 to 24 hours. As necessary, ventilation or stirring may also be carried out.

A transformant whose host is genus Bacillus is generally cultured at a temperature of approximately 30° C. to 40° C. for approximately 6 to 24 hours. As necessary, ventilation or stirring may also be carried out.

Examples of a medium used in the culture of a transformant whose host is yeast include: a Burkholder minimal medium [Bostian, K. L. et al., Proceedings of the National Academy of Sciences of the U.S.A. (Proc. Natl. Acad. Sci. U.S.A.), Vol. 77, 4505 (1980)]; and an SD medium containing 0.5% casamino acid [Bitter, G A. et al., Proceedings of the National Academy of Sciences of the U.S.A (Proc. Natl. Acad. Sci. U.S.A.), Vol. 81, 5330 (1984)]. The pH of such medium is preferably approximately p1-1 5 to 8. The culture is generally carried out at a temperature of approximately 20° C. to 35° C. for approximately 24 to 72 hours. As necessary, ventilation or stirring may also be carried out.

An example of a medium used in the culture of a transformant whose host is an insect cell or an insect is a Grace's Insect Medium (Grace, T. C. C., Nature, 195, 788 (1962)), to which additives such as an inactivated 10% bovine serum are added, as appropriate. The pH of such medium is preferably approximately pH 6.2 to 6.4. The culture is generally carried out at approximately 27° C. for approximately 3 to 5 days. As necessary, ventilation or stirring may also be carried out.

Examples of a medium used in the culture of a transformant whose host is an animal cell include: an MEM medium containing approximately 5% to 20% fetal bovine serum [Science, Vol. 122, 501 (1952)]; a DMEM medium [Virology, Vol. 8, 396 (1959)]; an RPMI 1640 medium [the Journal of the American Medical Association, Vol. 199, 519 (1967)]; and a 199 medium [Proceeding of the Society for the Biological Medicine, Vol. 73, 1 (1950)]. The pH of such medium is preferably approximately pH 6 to 8. The culture is generally carried out at a temperature of approximately 30° C. to 40° C. for approximately 15 to 60 hours. As necessary, ventilation or stirring may also be carried out.

As stated above, a DIVA protein can be generated within or outside of the cell of a transformant. The DIVA protein can be separated and purified from a culture obtained by culturing the aforementioned transformant in accordance with a known method.

Moreover, in the present invention, an antibody against DIVA or a partial peptide thereof (hereinafter abbreviated as an “anti-DIVA antibody” at times) can be produced. Such anti-DIVA antibody may be either a monoclonal antibody or a polyclonal antibody, as long as it has specific affinity for DIVA or the, peptide thereof. The antibody can be produced according to a known method for producing an antibody or an antiserum, using DIVA or a partial peptide thereof as an antigen.

### [Production of Monoclonal Antibody]

### (a) Production of Monoclonal Antibody-Producing Cells

DIVA or a partial peptide thereof is administered singly or together with a carrier, a diluent, and the like, to a site of a mammal, which is capable of producing antibody as a result of the administration. In order to enhance the ability of the mammal to produce antibody, a Freund's complete adjuvant or a Freund's incomplete adjuvant may also be administered. Administration is generally carried out once every 2 to 6 weeks, approximately 2 to 10 times in total. Examples of a mammal used herein include a monkey, a rabbit, a dog, a guinea pig, a mouse, a rat, a sheep and a goat. Of these, a mouse and a rat are preferably used.

For instance, some mammals having an antibody titer are selected from mammals immunized with an antigen, for example, from the immunized mice. Two to five days after the final immunization, the spleen or lymph node is collected from each mouse, and antibody-producing cells contained in the mouse are then fused with myeloma cells from the same type or different type of animals, so that monoclonal antibody-producing hybridomas can be prepared. The antibody titer in antiserum can be measured by allowing the below-mentioned labeled DIVA to react with the antiserum and then measuring the activity of a labeling agent binding to the antibody, for example. Fusion operations can be carried out by a known method such as the Kohler and Milstein method [Nature, 256, 495 (1975)]. As a fusion promoter, polyethylene glycol (PEG) or Sendai virus is used. Preferably, PEG is used.

Examples of myeloma cells used herein include the myeloma cells of mammals, such as NS-1, P3U1, SP2/0, and AP-1. Preferably, P3U1 is used. A preferred ratio between the number of antibody-producing cells (splenic cells) used and the number of myeloma cells used is approximately 1:1 to 20:1. PEG (preferably PEG1000 to PEG6000) is added in a concentration of approximately 10% to 80%, and the cells are then incubated at a temperature of 20° C. to 40° C., and preferably of 30° C. to 37° C., for 1 to 10 minutes, so that cell fusion can be efficiently carried out.

Monoclonal antibody-producing hybridomas can be screened by the following methods: a method comprising adding a hybridoma culture supernatant to a solid phase (e.g. a microplate) on which an antigen has been adsorbed directly or together with a carrier, and then adding an anti-immunoglobulin antibody (when cells used in cell fusion are mouse cells, an anti-mouse immunoglobulin antibody is used) or protein A, which has been labeled with a radioactive substance or enzyme, to the solid phase, so as to detect a monoclonal antibody binding to the solid phase; a method comprising adding a hybridoma culture supernatant to a solid phase on which an anti-immunoglobulin antibody or protein A has been adsorbed, and then adding DIVA that has been labeled with a radioactive substance or enzyme to the solid phase, so as to detect a monoclonal antibody binding to the solid phase; and other methods.

A monoclonal antibody can be selected according to a known method or a method equivalent thereto. Selection of a monoclonal antibody can be generally carried out in a medium used for animal cells, to which HAT (hypoxanthine, aminopterin, and thymidine) are added. The type of a medium used for selection of a monoclonal antibody and the growth thereof is not particularly limited, as long as it can be used for the growth of hybridomas. Examples of such medium used herein include an RPMI 1640 medium preferably containing 1% to 20%, and preferably 10% to 20% fetal bovine serum, a GIT medium containing 1% to 10% fetal bovine serum (Wako Pure Chemical Industries, Ltd.), and a serum-free medium used for the culture of hybridomas (SFM-101; Nissui Pharmachemical Co., Ltd.). The culture temperature is generally 20° C. to 40° C., and preferably approximately 37° C. The culture time is generally 5 days to 3 weeks, and preferably 1 week to 2 weeks. The culture can be generally carried out under 5% carbon dioxide. The antibody titer of a hybridoma culture supernatant can be measured in the same manner as the aforementioned measurement of the antibody titer in antiserum.

The thus obtained monoclonal antibody can be separated and purified in accordance with a known method such as a method for separating and purifying immunoglobulin [for example, a salting-out method, an alcohol precipitation method, an isoelectric point precipitation method, an electrophoresis method, an adsorption-desorption method using an ion exchanger (e.g. DEAE), an ultracentrifugation method, a gel filtration method, or a specific purification method, which comprises collecting only an antibody using an antigen-bound solid phase, or an active adsorber such as protein A or protein G, and then dissociating the bond, so as to obtain an antibody].

### [Production of Polyclonal Antibody]

A polyclonal antibody against DIVA or a partial peptide thereof can be produced according to a known method. For example, an immunogen (DIVA or a partial peptide thereof) is used, or a complex of such immunogen with a carrier protein is produced. Thereafter, a mammal is immunized with such immunogen or complex in the same manner as that in the aforementioned method for producing a monoclonal antibody, and an anti-DIVA antibody-containing material is then collected from the immunized mammal. Thereafter, separation and purification are performed to produce an antibody.

With regard to a complex of an immunogen with a carrier protein used in the immunization of a mammal, the type of the carrier protein and the mixing ratio between the carrier and the hapten are not particularly limited, as long as an antibody can be efficiently produced with respect to the hapten crosslinked with the carrier and then used for immunization. That is to say, the type of the carrier and the ratio between the carrier and the hapten to be crosslinked are not particularly limited. For instance, there is applied a method of coupling bovine serum albumin, bovine thyroglobulin, hemocyanin or the like with a hapten at a weight ratio of approximately 0.1:1 to 20:1, and preferably approximately 1:1 to 5:1.

In addition, for such coupling of a hapten with a carrier, there are used various condensing agents, such as glutaraldehyde, carbodiimide, or an active ester reagent containing maleimide active ester, a thiol group or a dithiopyridyl group.

A condensation product is administered singly or together with a carrier, a diluent, and the like, to a site of a mammal, which is capable of producing antibody. In order to enhance the ability of the mammal to produce antibody, a Freund's complete adjuvant or a Freund's incomplete adjuvant may also be administered. Administration is generally carried out once every 2 to 6 weeks, approximately 3 to 10 times in total.

A polyclonal antibody can be collected from the blood, ascites or the like of the mammal immunized by the aforementioned method, and preferably from the blood of the immunized mammal.

The polyclonal antibody titer in antiserum can be measured in the same manner as the aforementioned measurement of a monoclonal antibody titer in antiserum. The obtained polyclonal antibody can be separated and purified according to the same above method of separating and purifying immunoglobulin as in the case of the aforementioned separation and purification of a monoclonal antibody.

When a partial peptide of DIVA is used as an antigen, the position of the partial peptide on DIVA is not particularly limited. An example of such partial peptide is a polypeptide or oligopeptide having a partial amino acid sequence in a region well conserved among various types of homeotherms. In particular, when an antibody of interest is a neutralizing antibody, a partial peptide used as an antigen preferably contains all or a part of 81 kDa on the N-terminal side of DIVA.

The aforementioned anti-DIVA antibody can be used to measure the amount of DIVA or a salt thereof in a human or other homeotherms (for example, a rat, a mouse, a hamster, a rabbit, a sheep, a goat, a swine, a bovine, a horse, a cat, a dog, a monkey, a chimpanzee, a bird, etc.). Thus, such anti-DIVA antibody is useful as an agent for genetic diagnosis, such as an agent for diagnosing a decrease in the expression of the protein, an increase in the expression thereof, etc. For example, when an increase in the amount of DIVA in a sample is detected as a result of immunoassay, it can be diagnosed to have osteoarthritis, or to be highly likely to have osteoarthritis in future.

A substance capable of controlling (for example, suppressing) the activity of DIVA is useful for the prevention and/or treatment of osteoarthritis. Accordingly, the present invention provides a method for screening for an agent for preventing and/or treating osteoarthritis, which comprises administering a test substance to cells that express a gene encoding a protein consisting of the amino acid sequence of SEQ ID NO: 2 or an amino acid sequence substantially homologous thereto, and then selecting a substance that suppresses the expression of the gene.

For example, the present invention provides a method for screening for an agent for preventing and/or treating osteoarthritis, which is characterized in that it comprises comparing the expression of DIVA in cells having ability to produce such DIVA in the presence of a test substance with the same above expression of DIVA in the absence of a test substance.

Examples of a test substance include a protein, a peptide, a nonpeptidic compound; a synthetic compound, a fermented product, a cell extract, a plant extract, and an animal tissue extract. These may be either novel substances or known substances.

A test substance that suppresses the expression of DIVA in the aforementioned screening method can be selected as a “DIVA expression-suppressing substance.” Such DIVA expression-suppressing substance can be used as an agent for preventing and/or treating osteoarthritis.

The expression level of DIVA can also be measured at a transcription level, using a nucleic acid capable of hybridizing with a nucleic acid encoding DIVA under stringent conditions (that is, the aforementioned nucleic acid comprising the nucleotide sequence encoding DIVA or a portion thereof (hereinafter also referred to as “sense DIVA”) or a nucleotide sequence complementary to the nucleotide sequence encoding DIVA or a portion thereof (antisense DIVA)) to detect the mRNA thereof. Otherwise, such expression level can also be measured at a translation level, using the aforementioned anti-DIVA antibody to detect a protein (a peptide).

Accordingly, more specifically, the present invention provides:
(1) a method for screening for an agent for preventing and/or treating osteoarthritis, which is characterized in that it comprises culturing cells having ability to produce DIVA in the presence and absence of a test substance, then measuring the amounts of mRNAs encoding DIVA under the two above types of conditions, using sense or antisense DIVA, and then comparing the two types of results; and
(2) a method for screening for an agent for preventing and/or treating osteoarthritis, which is characterized in that it comprises culturing cells having ability to produce DIVA in the presence and absence of a test substance, then measuring the amounts of a DIVA proteins (peptides) under the two above types of conditions, using an anti-DIVA antibody, and then comparing the two types of results.

For example, the amount of mRNA of DIVA or a DIVA protein (peptide) can be specifically measured as described below.
(i) A test substance is administered to a normal- or disease-model nonhuman homeotherm (for example, a mouse, a rat, a rabbit, a sheep, a swine, a bovine, a cat, a dog, a monkey, a bird, or the like) a certain period of time before giving an agent, physical stimulation, or the like (30 minutes to 24 hours before, preferably 30 minutes to 12 hours before, and more preferably 1 hour to 6 hours before), or a certain period of time after giving an agent, physical stimulation, or the like (30 minutes to 3 days after, preferably 1 hour to 2 days after, and more preferably 1 hour to 24 hours after), or at a same time of giving an agent or physical stimulation. After a certain period of time has passed after the administration, nucleus pulposus, interspinal disk tissue, or the like is collected. The mRNA of DIVA expressed in cells contained in the obtained biological sample can be quantified, for example, by extracting mRNA from the cells or the like according to an ordinary method, and then by applying, for example, RT-PCR to extracted mRNA. Alternatively, such mRNA can also be quantified by the known Northern blot analysis. On the other hand, the amount of a DIVA protein can be quantified by Western blot analysis or various types of immunoassay methods as described in detail later.
(ii) A transformant, into which a nucleic acid encoding DIVA or a partial peptide thereof has been introduced, is produced by the aforementioned method. When the transformant is cultured by an ordinary method, a test substance is added to a medium. After completion of the culture for a certain period of time, the mRNA level of DIVA contained in the transformant or the amount of a protein (a peptide) can be quantified and analyzed.

Specific examples of a method for measuring the amount of DIVA in the aforementioned screening method include: (i) a method, which comprises allowing an anti-DIVA antibody to competitively react with a sample solution and labeled DIVA, and then detecting the labeled DIVA that binds to the antibody, so as to quantify the DNA contained in the sample solution; and (ii) a method, which comprise allowing a sample solution to simultaneously or continuously react with an anti-DNA antibody insolubilized on a carrier and another anti-DIVA antibody labeled, and then measuring the amount (activity) of a labeling agent on the insolubilized carrier, so as to quantify the DIVA contained in the sample solution.

In the quantification method described in (ii) above, the two types of antibodies desirably recognize different portions of DIVA. For example, when one antibody recognizes the N-terminal portion of DIVA, then, an antibody reacting with the C-terminal portion of DIVA can be used as the other antibody.

Examples of a labeling agent used in a measurement method using such labeling substance include a radioisotope, an enzyme, a fluorescent substance, and a luminescent substance. Examples of such radioisotope include [¹²⁵I], [¹³¹I], [³H], and [¹⁴C]. As an enzyme used herein, a stable enzyme having large specific activity is preferable. Specific examples of such enzyme include β-galactosidase, β-glucosidase, alkaline phosphatase, peroxidase, and malate dehydrogenase. Specific examples of a fluorescent substance used herein include fluorescamine and fluorescein isothiocyanate. Specific examples of a luminescent substance used herein include luminol, a luminol derivative, luciferin, and lucigenin. Furthermore, a biotin-(strepto)avidin system can be used for the binding of an antibody or an antigen with a labeling agent.

When DIVA is localized in a cell, a cell disintegrated solution obtained by suspending cells in an appropriate buffer and then disintegrating the cells by an ultrasonic treatment or freezing and thawing is used as a sample solution. When DNA is secreted outside of a cell, a cell culture supernatant is used as a sample solution. After DIVA has been separated and purified from such disintegrated solution or culture supernatant, it may be quantified, as necessary. Moreover, an intact cell may be used as a sample, as long as a labeling agent can be detected.

A method for quantifying DIVA using an anti-DIVA antibody is not particularly limited. Any type of measurement method may be used, as long as it is a measurement method, which comprises detecting the amount of an antibody, an antigen, or an antibody-antigen complex corresponding to the amount of an antigen in a sample solution by chemical or physical means, and then calculating such amount based on a standard curve prepared using a standard solution containing a known amount of antigen. For example, nephelometry, a competitive method, an immunometric method, and a sandwich method are preferably used. In terms of sensitivity and specificity, a sandwich method as described later is preferably used, for example.

In order to insolubilize an antigen or an antibody, there may be used either physical adsorption, or chemical bond that is generally used to insolubilize and/or immobilize a protein, an enzyme, or the like. Examples of a carrier include: insoluble polysaccharides such as agarose, dextran or cellulose; synthetic resins such as polystyrene, polyacrylamide or silicon; and glass.

In the sandwich method, a sample solution is allowed to react with the insolubilized anti-DIVA antibody (a first reaction), and the sample solution is then allowed to react with another anti-DIVA antibody labeled (a second reaction). Thereafter, the amount or activity of a labeling agent on an insolubilized carrier is measured, so as to quantify DIVA in the sample solution. The first reaction and the second reaction may be carried out in the reverse order, or the two reactions may be carried out simultaneously. Otherwise, the two reactions may also be carried out while shifting time. The same labeling agent and the same insolubilization method as those described above may be applied. In addition, in an immunoassay that utilizes a sandwich method, an antibody used as a solid-phased antibody or as a labeled antibody is not necessarily of one type. For the purpose of the improvement of measurement sensitivity and the like, a mixture of two or more types of antibodies may also be used.

The anti-DIVA antibody may also be used for measurement systems other than the sandwich method, for example, for a competitive method, an immunometric method, nephelometry, or the like.

In the competitive method, DNA contained in a sample solution and labeled DIVA are allowed to competitively react with an antibody, and an unreacted labeled antigen (F) is then separated from a labeled antigen (B) binding to the antibody (B/F separation). Thereafter, the amount of B or F labeled is measured, so that the DIVA contained in the sample solution can be quantified. The present reaction method includes: a liquid phase method comprising conducting B/F separation using a soluble antibody and also using polyethylene glycol, a secondary antibody against the aforementioned antibody (primary antibody), or the like; and a solid phase method, using a solid phased antibody as a primary antibody (a direct method), or using a soluble antibody as a primary antibody and a solid-phased antibody as a secondary antibody (an indirect method).

In the immunometric method, DIVA contained in a sample solution and solid-phased DIVA are allowed to competitively react with a certain amount of labeled antibody, and a solid phase is then separated from a liquid phase. Otherwise, DIVA contained in a sample solution is allowed to react with an excessive amount of labeled antibody, and solid-phased DIVA is then added thereto, so that un reacted labeled antibody is allowed to bind to a solid phase. Thereafter, the solid phase is separated from a liquid phase. Subsequently, the labeled amount of either one phase is measured, and the amount of an antigen contained in the sample solution is quantified.

In addition, in nephelometry, the amount of an insoluble precipitate generated as a result of an antigen-antibody reaction in a gel or in a solution is measured. Even when the amount of DIVA in a sample solution is extremely small and only a small amount of precipitate is obtained, laser nephelometry using laser scattering and the like are preferably used.

In order to apply these immunological measurement methods to the quantification method of the present invention, it is not necessary to set special conditions, operations, and the like. Persons skilled in the art may construct a DIVA measurement system by combining ordinary conditions and operations for each method with their general technical consideration. For the details of such general technical means, review papers, textbooks, and the like can be referred.

For example, the following publications can be referred: Hiroshi Irie, “Radioimmunoassay” (Kodansha Ltd., 1974); Hiroshi Irie “Sequel to Radioimmunoassay” (Kodansha Ltd., 1979); Eiji Ishikawa et al., “Koso Meneki Sokuteiho (Enzyme Immunoassay)” (Igaku-Shoin Ltd., 1978); Eiji Ishikawa et al., “Koso Meneki Sokuteiho (Enzyme Immunoassay) (2^{nd} edition)” (Igaku-Shoin Ltd., 1982); Eiji Ishikawa et al., “Koso Meneki Sokuteiho (Enzyme Immunoassay) (3^{rd} edition)” (Igaku-Shoin Ltd., 1987); Methods in ENZYMOLOGY, Vol. 70 (Immunochemical Techniques (Part A)); same publication, Vol. 73 (Immunochemical Techniques (Part B)); same publication, Vol. 74 (Immunochemical Techniques (Part C)); same publication, Vol. 84 (Immunochemical Techniques (Part D: Selected Immunoassays)); same publication, Vol. 92 (Immunochemical Techniques (Part E: Monoclonal Antibodies and General Immunoassay Methods)); and same publication, Vol. 121 (Immunochemical Techniques (Part I: Hybridoma Technology and Monoclonal Antibodies)), (which are published from Academic Press).

As described above, using an anti-DIVA antibody, the amount of DIVA produced in cells can be quantified with high sensitivity.

In the above-described screening method, a substance that suppresses the expression level of DIVA (the mRNA level or the amount of the protein (peptide)) can be selected as a DNA expression-suppressing substance. Such DIVA expression-suppressing substance can be used as an agent for preventing and/or treating osteoarthritis (for example, knee osteoarthritis).

Moreover, the present invention provides an agent for preventing and/or treating osteoarthritis, which comprises a protein (DIVA) inhibitor consisting of the amino acid sequence of SEQ ID NO: 2 or an amino acid sequence substantially homologous therewith.

The DIVA inhibitor used in the present invention includes a substance for inhibiting the expression of DIVA, a substance that acts on DNA to inhibit the activity or functions of the DIVA, and the like.

Such substance for suppressing the expression of DNA includes substances utilizing RNAi, an antisense method, or a ribozyme method. Thus, the type of such substance is not particularly limited. Among others, siRNAs utilizing RNAi are preferable. A substance that acts on DIVA to inhibit the activity or functions of the DIVA includes a low molecular weight compound, an antibody, and the like.

RNAi (RNA interference) is a phenomenon whereby double-stranded RNA introduced into a cell suppresses the expression of a gene having the same sequence. Specific examples of a substance for inhibiting the expression of DIVA due to RNAi include siRNA, shRNA, and the like, which are described below.

siRNA is an abbreviated name for short interfering RNA, which is double-stranded RNA having a length of approximately 21 to 23 nucleotides. The form of siRNA is not particularly limited, as long as it causes RNAi. Examples of such siRNA include: siRNA obtained by chemical synthesis, biochemical synthesis, or synthesis occurring in an organism; and short-chain double-stranded RNA consisting of 10 or more base pairs obtained by decomposing double-stranded RNA consisting of approximately 40 or more nucleotides in vivo. The sequence of siRNA is preferably 100% identical to the partial sequcne of mRNA of DIVA. However, the two above sequences are not necessarily 100% identical to each other.

Preferably, a region having homology between the nucleotide sequence of siRNA and the nucleotide sequence of the DIVA gene does not contain the translation initiation region of the DIVA gene. The region having such homology is preferably apart from the translation initiation region of the DIVA gene by 20 nucleotides, and more preferably by 70 nucleotides. An example of the sequence having such homology may be a sequence around the 3′-terminus of the DIVA gene.

As a substance that inhibits the expression of DIVA due to RNAi, dsRNA consisting of approximately 40 or more nucleotides that generates siRNA and the like may be used. For example, there can be used RNA containing a double-stranded portion, which comprises a sequence having homology of approximately 70% or more, preferably 75% or more, more preferably 80% or more, further preferably 85% or more, still further preferably 90% or more, particulary preferably 95% or more, and most preferably 100%, with a portion of the nucleic acid sequence of the DIVA gene, or a modified body thereof. A sequence portion having homology consists of generally at least 15 nucleotides, preferably approximately 19 or more nucleotides, more preferably at least 20 nucleotides, and further preferably 21 or more nucleotides.

As a substance that inhibits the expression of DIVA due to RNAi, shRNA (short hairpin RNA) having a short hairpin structure with a projection at the 3′-terminus can also be used. shRNA is a molecule consisting of approximately 20 or more base pairs. Since such shRNA is single-stranded RNA partially comprising a palindromic nucleotide sequence, it adopts a double-stranded structure in the molecule, so as to have a structure such as a hairpin. In addition, such shRNA preferably has a 3′-protruding end. The length of such double-stranded portion is not particularly limited. It is preferably 10 or more nucleotides, and more preferably 20 or more nucleotides. Herein, the 3′-protruding end is preferably DNA, more preferably DNA consisting of at least 2 nucleotides, and further preferably DNA consisting of 2 to 4 nucleotides.

The substance that inhibits the expression of DIVA due to RNAi may be artificially chemically synthesized. Alternatively, it may also be produced by synthesizing, in vitro, RNA from DNA having a hairpin structure formed by reversely ligating the DNA sequence of a sense strand to the DNA sequence of an antisense strand, using T7 RNA polymerase. When such RNA is synthesized in vitro, antisense and sense RNAs can be synthesized from template DNA, using T7 RNA polymerase and a T7 promoter. These RNAs are annealed to each other in vitro, and the obtained product is then introduced into a cell. As a result, RNAi takes place, and the expression of DIVA is thereby suppressed. The introduction of the obtained product into a cell can be carried out by a calcium phosphate method, methods using various types of transfection reagents (for example, oligofectamine, Lipofectamine, lipofection, etc.), and other methods.

As a substance that inhibits the expression of DIVA due to RNAi, there may also be used an expression vector containing a nucleic acid sequence encoding the aforementioned siRNA or shRNA. Further, a cell containing the aforementioned expression vector may also be used. The type of the aforementioned expression vector or cell is not particularly limited. An expression vector or a cell, which has already been used as a pharmaceutical agent, is preferable.

The administration route of the agent for preventing and/or treating osteoarthritis of the present invention, which comprises a protein inhibitor consisting of the amino acid sequence of SEQ ID NO: 2 or an amino acid sequence substantially homologous therewith, is not particularly limited. The aforementioned agent may be administered via either an oral administration route or a parenteral administration route (for example, intravenous administration, intramuscular administration, subcutaneous administration, intradermal administration, mucosal administration, local administration to affected area, skin administration, etc.). The form of a pharmaceutical agent suitable for oral administration includes a solid and a liquid. The form of a pharmaceutical agent suitable for parenteral administration includes an injection, drops, a suppository, an external preparation, and the like. Pharmaceutically acceptable additives may be added to the agent for preventing and/or treating osteoarthritis of the present invention, as necessary, depending on the form of the agent. Specific examples of such pharmaceutically acceptable additive include an excipient, a binder, a disintegrator, a lubricant, an antioxidant, a preservative, a stabilizer, an isotonizing agent, a coloring agent, a corrective, a diluent, an emulsifier, a suspending agent, a solvent, a filler, a thickener, a buffer, a delivery vehicle, a diluent, a carrier, an excipient, and/or a pharmaceutical adjuvant.

The agent for preventing and/or treating osteoarthritis of the present invention that is in the form of a solid agent for oral use can be prepared as a tablet, granules, powders, or a capsule, by adding an excipient to a DIVA inhibitor as an active ingredient, and further adding thereto, as necessary, pharmaceutical additives such as a binder, a disintegrator, a lubricant, a coloring agent, or a corrective, followed by the application of an ordinary method. The agent for preventing and/or treating osteoarthritis of the present invention that is in the form of a liquid agent for oral use can be prepared as a liquid agent for internal use, a syrup, an elixir agent, or the like, by adding one or two or more types of pharmaceutical additives such as a corrective, a stabilizer, or a preservative, to a DIVA inhibitor as an active ingredient, followed by the application of an ordinary method.

A solvent used to prepare the agent for preventing and/or treating osteoarthritis of the present invention as a liquid agent may be either an aqueous or nonaqueous solvent. Such liquid agent can be prepared by a method publicly known in the present field. For example, an injection can be prepared by dissolving the present agent in a solvent including a normal saline, a buffer such as PBS, sterilized water, or the like, then conducting filtration sterilization using a filter, and then filling an aseptic container (for example, an ampule) with the resultant solution. This injection may comprise a commonly used pharmaceutical carrier, as necessary. In addition, an administration method using a noninvasive catheter may also be applied. The carrier used in the present invention includes a neutrally buffered normal saline, a normal saline containing serum albumin, or the like.

The type of the gene delivery of the siRNA of DIVA or an siRNA expression vector is not particularly limited, as long as it enables the expression of RNA encoding the siRNA of DIVA or the siRNA expression vector in tissues applied. For example, it is possible to apply gene introduction using a viral vector or liposome. Examples of such viral vector include animal viruses such as retrovirus, vaccinia virus, adenovirus, or Semliki Forest virus.

The substance that inhibits the expression of DIVA due to RNAi may be directly injected into the organs, tissues, or the like of an organism.

The dosage of the agent for preventing and/or treating osteoarthritis of the present invention may be determined by persons skilled in the art, while taking into consideration intended use, the severity of disease, the age of a patient, body weight, sex, anamnesis, the type of active ingredient, and the like. When the active ingredient of the agent for preventing and/or treating osteoarthritis of the present invention is a substance that inhibits the expression of DIVA due to RNAi, for example, the dosage (the amount of the active ingredient) of the agent is approximately 0.1 ng/kg to approximately 100 mg/kg, and preferably approximately 1 ng/kg to approximately 10 mg/kg, per adult. When the present agent is administered in the form of a viral or non-viral vector, the dosage is generally 0.0001 to 100 mg, preferably 0.001 to 10 mg, and more preferably 0.01 to 1 mg.

With regard to administration frequency, the agent for preventing and/or treating osteoarthritis of the present invention may be administered once per day to once per several months, for example. When the substance that inhibits the expression of DIVA due to RNAi is used, it generally exhibits effects 1 to 3 days after administration. Thus, it is preferable to administer the agent at a frequency of once every day to once every three days. When an expression vector is used, there may also be a case in which the agent is administered approximately once a week.

The present invention will be more specifically described in the following examples. However, these examples are not intended to limit the scope of the present invention.

### Examples

### (A) Materials and Methods

### (1) Subjects

Patients suffering from knee osteoarthritis and control patients (set A and set B), and patients suffering from hip osteoarthritis, were registered. Osteoarthritis was diagnosed on the basis of clinical and radiographic findings. The diagnosis criteria of knee osteoarthritis and hip osteoarthritis were described previously (Ikeda, T., A. Mabuchi, et al. (2001). J Hum Genet 46(9): 538-43; and Mabuchi, A., T. Ikeda, et al. (2001). J Hum Genet 46(8): 456-62). These knee osteoarthritis populations include the individuals with two or higher JSN (joint space narrowing) grade. Further, population-based cohorts from inhabitants of Odai and Minami-ise town (previous names were Miyagawa village and Nansei town) in Mie prefecture in Japan was registered as set C. Each subject of the cohort into the knee osteoarthritis population and the control population was classified according to the radiographic findings. The criteria of knee osteoarthritis for these cohort were two or higher Kellegren-Lawrence grade (Kellgren et al., 1957) for the individuals in Odai town, and two or higher JSN grade for the individuals in Minami-ise town. Clinical parameters of the populations in this study are shown in Table 11, including numbers, male-female ratio, mean age and mean body mass index (BMI). Genomic DNA was extracted from peripheral blood leukocytes of affected individuals and controls using standard protocols.

Osteoarthritis cartilage was obtained from knee during total knee arthroplasty (7 samples). Normal cartilage was obtained from femoral heads of control individuals during surgery for femoral neck fracture (8 samples). None of the control individuals had a clinical history or any radiographic sign of hip osteoarthritis. Written informed consent was obtained from each subject as approved by the ethical committees of the SNP Research Center at RIKEN and participating clinical institutes.

### (2) Genotyping of SNPs

SNPs were genotyped using the multiplex PCR-based Invader assay (Ohnishi, Y., T. Tanaka, et al. (2001). J Hum Genet 46(8): 471-7.)(Third Wave Technologies), TaqMan SNP genotyping assays (Applied Biosystems) or by direct sequencing of PCR products using ABI 3700 DNA analyzers (Applied Biosystems) according to the manufacturers' protocols.

### (3) Statisitical Analysis

Haplotype frequencies were estimated by EM-algorithm (Excoffier, L. and M. Slatkin (1995). Mol Biol Evol 12(5): 921-7). Statistical analyses was carried out for the association, haplotype frequencies and Hardy-Weinberg equilibrium and linkage disequilibrium coefficients (D′ and r²) was calculated using Haploview software 3.32 (Barrett et al., 2005) and Excel 2004 (Microsoft).

### (4) Cell Culture and RNA Extraction

HEK293, chondrogenic HCS-2/8 and OUMS-27 cells were cultured in Dulbecco's modified Eagle's medium containing 10% fetal bovine serum (FBS) at 37° C. under 5% CO₂. Normal human articular chondrocyte (NHAC-kn; Cambrex) was purchased and maintained in the supplied medium of the kit. mRNA for RACE, RT-PCR and northern blotting was extracted from cultured cells using FastTrack 2.0 Kit (Invitrogen). Total RNA was extracted from cultured cells using Isogen (Nippongene) and SV Total RNA Isolation System (Promega). Total RNA from cartilage tissue was extracted using RNeasy Lipid Tissue Kit (Qiagen). All protocols were according to the manufacturers' instructions.

### (5) RACE, RT-PCR and Real-Time PCR

5′- and 3′-RACE was performed using SMART RACE cDNA Amplification Kit (Clontech) according to the manufacturer's protocol. 1 μg mRNA extracted from normal human articular cartilage was used for the production of RACE template. cDNA of various tissues except cartilage was obtained from Multiple Tissue cDNA Panels (Clontech). Cartilage and cell line cDNA for RT-PCR and real-time PCR was synthesized using Multiscribe reverse transcriptase and oligo-dT primer (Applied Biosystems). Quantitative real-time PCR was performed using an ABI PRISM 7700 sequence detector with Quantitect SYBR Green PCR Kit (Qiagen) in accordance with the manufacturers' instruction.

Primers used in the present experiment are as follows:

(SEQ ID NO: 3)

| | |
|---|---|
| 5′-RACE: | ctcactgctgacgttgaagctgtttacc |

(SEQ ID NO: 4)

| | |
|---|---|
| Nested 5′-RACE: | gtgtccaccagaaacacaacatctccc |

(SEQ ID NO: 5)

| | |
|---|---|
| 3′-RACE: | tgggcagagctcagggaaattgccagta |

(SEQ ID NO: 6)

| | |
|---|---|
| Nested 3′-RACE: | agaagctgcggcaggaactctgtgatac |

(SEQ ID NO: 7)

| | |
|---|---|
| Real-time PCR, forward: | gcggcaggaactctgtgata |

(SEQ ID NO: 8)

| | |
|---|---|
| Real-time PCR, reverse: | atgtcaagccccaagatgac |

### (6) Luciferase Assay

For measurement of the promoter assay, the DNA fragment corresponding to nucleotide −342 to 34 of DIVA (wherein the transcription start point was defined as +1) was amplified by PCR using genomic DNA as template, and was cloned into pGL3 basic vector (Promega) in the 5′-3′ orientation. Cells (5×10⁴) were transfected with 0.4 μg of the constructed pGL3 vector and 4 ng of pRL-TK vector as an internal control, using TransIT-293 (for HEK293) or TransIT-LT1 (for other cell lines) reagent (Mirus). After 48 hours, the cells were collected, and luciferase activity was measured using the PicaGene Dual Sea Pansy system (Toyo Ink).

### (7) Northern Blotting

The cDNA fragment corresponding to nucleotide 510 to 1517 of DIVA was cloned into pCR2.1TOPO vector (Invitrogen). The DIG-labeled probe was synthesized from the constructed vector using DIG RNA Labeling Kit (Roche). 4 ug of normal human articular cartilage, HCS-2/8 and OUMS-27 mRNA were electorophoresed. Transfer to membrane, hybridization and detection of bands were performed using DIG Easy Hyb and DIG Wash and Block Buffer set (Roche) according to the manufacturer's instructions.

### (8) Immunoprecipitation

The whole coding sequence of DIVA was cloned into pTriEx4 vector (Novagen). This vector expresses amino-terminal S-tagged DIVA in mammalian cells. The vector or pTriEx4 containing no insert (which expresses S-tagged artificial control protein) was transiently transfected into HCS-2/8 or HEK293 cells. Cell lysates were harvested and the immunoprecipitation was performed using S-protein agarose (Novagen) according to the manufacturer's instruction. After SDS-PAGE, target protein bands were analyzed by MALDI/TOF mass spectrometry at APRO Life Science, or by western blotting using anti-β-tubulin antibody (Santa Cruz) and S-protein-HRP (Novagen).

### (9) Recombinant Protein and Solid-Phase Binding Assay

Rosetta (DE3) pLacl (Novagen) was transformed with pTriEx4-DIVA and cultured in Overnight Express Autoinduction System (Novagen). DIVA recombinant protein was extracted using BugBuster Protein Extraction Reagent (Novagen), and refolded from an insoluble fraction using Protein Refolding Kit (Novagen). Maxisorp ELISA plate (Nunc) wells were coated with 100 μl of 50 μg/ml recombinant S-tagged DIVA protein in 50 mM NaHCO₃ buffer (pH 9.6) at 4° C. overnight. Then, the wells were blocked with 100 μl of 5% bovine serum albumin (BSA) in PBS for one hour, and the wells were incubated in a PBS solution (total volume of 100 μl) of 5% BSA added with 5 μg of bovine tubulin (Cytoskeleton) at 4° C. overnight. The wells were washed three times with TBST (20 mM Tris-HCl (pH 7.5), 137 mM NaCl and 0.05% Tween 20) and incubated with β-tubulin-HRP antibody (Santa Cruz) for one hour. After washing five times with TBST, the bound tubulin was assayed using TMB Peroxidase EIA Substrate Kit (Biorad).

### (10) Production of DIVA Antibody

Using the entire-length or partial DIVA E.coli recombinant protein produced in (9) above as an antigen, an antibody was produced. First, GeneFrontier Corporation was asked to produce a monoclonal antibody by a screening procedure using an HcCAL phage library. In addition, Peptide Institute, Inc. was asked to produce a polyclonal antiserum by a method of immunizing a rabbit. It was confirmed by the two above companies that both the antibody and the antiserum reacted with an antigenic protein by ELISA.

### (B) Results

### (1) Genomewide Screening

Genome-wide association study was performed using 94 cases with knee osteoarthritis and 658 controls (set A) (Ozaki, K., Y. Ohnishi, et al. (2002). Nat Genet 32(4): 650-4). 99,295 single nucleotide polymorphisms (SNPs) selected from the JSNP database were genotyped (Hags, H., R. Yamada, et al. (2002). J Hum Genet 47(11): 605-10). After checking quality of the data, the results of 79,763 SNPs were compared between cases and controls. χ² tests were performed for genotype, dominant, recessive and allele frequency models, and 2,153 SNPs showing P values under 0.01 in any of the four models were identified. Further, these 2,153 SNPs were genotyped using independent populations which consisted of 646 knee osteoarthritis cases and 631 controls (set B). It was revealed that rs3773472 in an intron of SH3BP5 shows strong association (P=0.000017 for allele frequency model; Table 1). This result remained significant after Bonferroni's correction for multiple testing (0.000017 X 2,153=0.037). Therefore, it was decided to examine SNPs in the region around rs3773472.

### (2) Linkage Disequilibrium Analysis and Tagging

The International HapMap Project database (http://hapmap.org, release #21a) was searched, and SNPs with D′ value of >0.7 to rs3773472 and with a minor allele frequency of >0.1 were selected. This linkage disequilibrium (LD) block around rs3773472 contained 40 HapMap SNPs, two validated genes (SH3BP5 and CAPN7) and one predicted gene (LOC344875). Next, 12 tag SNPs (including rs3773472) that covered all these 40 SNPs with r² value of >0.9 were selected. The tag SNPs were genotyped using the population set B, and SNP which is more associated with knee osteoarthritis than rs3773472 was revealed (rs7639618, P=7.3×10⁻⁸ for the allele frequency model; Table 2). An odds ratio for the susceptibility allele of rs7639618 was 1.54 (95% confidence interval=1.32−1.81).

### (3) Check for Confounding Factors

Effects of confounding factors such as age, body mass index (BMI) and sex were checked to evaluate whether they could make a pseudo-positive association with knee osteoarthritis. There was no significant difference in mean age, BMI and sex distribution between genotypes of rs7639618 (Table 3). An effect of population stratification was also examined using a genomic control method (Pritchard, J. K. and N. A. Rosenberg (1999). Am J Hum Genet 65(1): 220-8; and Freedman, M. L., D. Reich, et al. (2004). Nat Genet 36(4): 388-93). It was found that it is an unlikely explanation for the positive association of rs7639618 (Table 4).

### (4) Replication Study

To confirm the association, a replication study was performed using an independent population cohort, which is divided by the findings of knee radiographs to knee osteoarthritis and control population. rs7639618 was genotyped using 242 knee osteoarthritis cases and 485 controls (set C), and the result was also significant (P=0.038; Table 5). Next, it was examined whether rs7639618 was associated with hip osteoarthritis as well as knee osteoarthritis. 803 hip osteoarthritis cases were recruited, rs7639618 was genotyped, and the result was compared to the set B control. The association of rs7639618 with hip osteoarthritis was only marginal (P=0.062; Table 6) as to significant difference. Because acetabular dysplasia of the hip is a major predisposing factor of hip osteoarthritis in Japanese (Nakamura et al., 1989), a stratification analysis was performed by presence of acetabular dysplasia. Significant association was found between rs7639618 showed and the hip osteoarthritis cases without acetabular dysplasia (P=0.037).

### (5) Identification of DIVA

In the NCBI genome database (build 36.2), rs7639618 is inside LOC344875 gene. The RefSeq transcript of LOC344875 (XM_{—}497913) is based on in silico predictions and ESTs only. Therefore, it was decided to examine the full sequence of the expressed transcript by RACE and RT-PCR using normal human articular chondrocyte cDNA as a template, and a novel transcript, which was different from XM_{—}497913 was found (FIG. 1). Multiple transcription start site (TSS) was found by 5′-RACE, but the TSS showed in FIG. 1 was the major one, because the upstream 1,000-bp sequence of this TSS was predicted to be promoter region by the PROMOTER SCAN program (Prestridge, 1995). It was also confirmed that the upstream region of this TSS had the promoter activity by luciferase assays (FIG. 2). This transcript was 2,250-bp long and contained 1,098-bp open reading frame (ORF). The predicted protein consisted of 276 amino acids. The programs for protein motif analysis, Pfam (Finn et al., 2006) and PSORT (Nakai et al., 1999) predicted that this protein had no signal peptide and two domains which were homologous with von Willebrand factor domain A (VWA domain). The present inventors named this new gene DIVA, after Dual Intracellular Von Willebrand factor domain A. Because all 17 HapMap SNPs that linked to rs7639618 with r²>0.9 were in and around this DIVA region, it was judged that DIVA was likely to be the associated gene with osteoarthritis rather than SH3BP5 and CAPN7. To confirm the expression and the size of the DIVA transcript, northern blotting was performed using chondrocyte mRNA, showing the band that corresponded to the predicted size (FIG. 3a).

### (6) Expression Profile of DIVA

To characterize DIVA, DIVA expression was examined in various human tissues using real-time PCR. The highest levels of DNA expression was detected in normal and osteoarthritis cartilage tissues (FIG. 3b). This observation suggested that the function of DIVA has relations with cartilage tissue.

### (7) Searching for Disease-Causing SNP

To locate the functional, osteoarthritis-associated SNP, SNPs in and around all exons of DIVA were searched by direct sequencing using genomic DNA from 48 individuals with knee osteoarthritis. Four more SNPs were found in this experiment in addition to 21 SNPs in the HapMap database (Table 7). Pairwise r² value was calculated using all these 25 SNPs in the DIVA region, and the tag SNPs with r²>0.95 were selected. In addition to the four SNPs which were already genotyped (rs826428, rs353093, rs7639618 and rs618762), three SNPs were selected and genotyped. Among these tag SNPs, rs9864422 was more associated with knee osteoarthritis (P=2.4×10⁻⁸ for allele frequency model; Table 8) than rs7639618. There was no other SNP that linked to rs9864422 with r²>0.95.

### (8) Haplotype Analyses

Haplotype association analyses was performed using the first tag SNP set of 12 SNPs in the whole linkage disequilibrium block and the second tag SNP set of 7 SNPs in the DIVA region (Table 9). None of the haplotype showed the lower P value than that of rs9864422 or rs7689618. This result suggests that, among SNPs whose genotypes had been analyzed to date, there were no SNPs that were associated with knee osteoarthritis more strongly than rs9864422 or rs7689618 were. The most associated SNP rs9864422 and two highly associated missense SNPs (rs7639618 and rs11718863) were chosen as candidates of the osteoarthritis-associated SNP for further analyses of allelic functional difference.

### (9) Binding to Tubulin and Allelic Difference of the Missense SNPs

First, the function of the missense SNPs was evaluated. rs 11718863 and rs7639618 yielded three haplotypes, two of which were highly associated (Table 10). To find binding partners of DIVA, immunoprecipitaion analysis was performed. After transfection of DIVA and purification using S-tag, a unique band that could be observed when S-tag-DIVA was expressed was identified (FIG. 4a). MALDI/TOF (matrix-assisted laser desorption/ionization-time of flight) mass spectrometry analysis revealed that the band corresponded to β-tubulin. The binding between DIVA and tubulin was confirmed by immunopreciptation and western blotting (FIG. 4b). Next, the binding power between tubulin and isoforms of DIVA was assessed. S-tagged recombinant proteins that correspond to four haplotypes of the two missense SNPs were generated (FIG. 4c). Solid-phase binding assays was performed using these recombinant proteins and tubulin. All four isoforms of DIVA bound to tubulin, and the binding power of DIVA-169Y-260C was significantly weaker than other three isoforms (FIG. 4d). DIVA-169Y-260C was also the isoform that over-expressed in knee osteoarthritis population (Table 10).

### (10) Functions of DIVA Gene in Human Chondrocytes

To examine the functions of the DIVA gene in human chondrocytes, using a human chondrocyte line, OUMS-27 cells, the action of the DIVA gene on the expression of initial differentiation marker genes of cartilage, such as type-II collagen (COL2A1) or aggrecan (AGC1), was analyzed.

First, using DIVA-specific siRNA, the functions of intrinsic DIVA were examined. DIVA-specific siRNA (Si3: wherein GUAGACAGUUCAACUAGCATT (sense) (SEQ ID NO: 9) and UGCUAGUUGAACUGUCUACTT (antisense) (SEQ ID NO: 10) were annealed, and the TT at 3′ was overhung) was designed using an siRNA design support system in the homepage of Takara Shuzo Co., Ltd. (http://www.takara-bio.co.jp/mai/intro.htm). As control siRNA, a sequence matched with neither DIVA nor other genes was designed and used. Cells were dispersed on a 12-well plate to a concentration of 5×10⁵ cells/well. The cells were then cultured in a medium containing 10% FBS and penicillin/streptomycin for 24 hours. Thereafter, the cells were transfected with siRNA, using TranslT-TKO (Takara Shuzo Co., Ltd.). After completion of the culture for 24 hours, total RNA was extracted from the cells, and the expression of a type-II collagen gene and that of an aggrecan gene were quantified by a real-time PCR method. As a result, it was found that the DIVA-specific siRNA significantly increased the expression of the type-II collagen gene and that of the aggrecan gene (FIG. 5). Accordingly, the intrinsic DIVA was considered to negatively control the differentiation of the cartilage in the OUMS-27 cells.

In order to confirm the aforementioned functions of DIVA, the OUMS-27 cells were forced to express the DIVA gene, and the action of the DIVA gene on the expression of initial differentiation marker genes of cartilage, such as type-II collagen (COL2A 1) or aggrecan (AGC1), was analyzed. The time course after transfection with 10 pg of a vector was analyzed and was then optimized. Twenty-four hours later, the expression of DIVA and the expressions of the cartilage marker genes were quantified by a real-time PCR method. As a result, it was found that the expressions of cartilage marker genes were significantly decreased by the expression of the DIVA gene (FIG. 6).

As stated above, it was discovered that DIVA has the function of negatively controlling cartilage differentiation. The aforementioned results demonstrated that a compound for suppressing the expression or action of DIVA would be likely to promote cartilage differentiation in the articular cartilage, and that such compound becomes a therapeutic agent for osteoarthritis.

### (11) Western Blotting Using DIVA Antibody

Using a monoclonal antibody and a polyclonal antiserum produced using a DIVA Escherichia coli modified protein as an antigen, Western blotting was carried out. 100 ng of an antigenic protein was electrophoresed by SDS-PAGE, and it was then transferred onto a PVDF membrane. Thereafter, it was blocked with 10% BSA or Block-Ace. The monoclonal antibody was reacted at a concentration of 5 mg/ml, and the polyclonal antibody was 10,000 times diluted and was then reacted. The results are shown in FIG. 7. In both cases, a band could be obtained at a predicted size, and thus it was demonstrated that an antibody against an antigen of interest could be produced.

### (C) Consideration

### (1) Predicted Function of DIVA

DIVA protein had two domains that are homologous with VWA domain. VWA domains are usually involved in cell adhesion and protein-protein interaction. Mutations in VWA domains of MATN3 (OMIM 602109) cause osteoarthritis and osteochondrodysplasia. Although the majority of VWA domain-containing proteins are extracellular, some are intracellular and involved in functions such as transcription, DNA repair, ribosomal and membrane transport (Whittaker et al., 2002). Taken account of these things, It is predicted that DIVA binds to proteins including tubulin and engages in some functions such as an assist of transport intracellularly.

### (2) The Binding Between DIVA and Tubulin

In the aforementioned examples, it was proved that DNA interacts with tubulin and its binding power was affected by the alleles of the highly associated two missense SNPs. These SNPs lies in the predicted VWA domain, so it is highly possible that these SNPs affect the binding of DIVA. Because the overrepresented DNA isoform in osteoarthritis showed weaker binding to tubulin, there is a possibility that the interaction between DIVA and tubulin protects joints from onset of osteoarthritis. Only 169Y-260C isoform of DIVA showed weaker interaction with tubulin, and other three isoforms had the similar level of interaction. Thus it is considered that that two SNPs cooperate in binding between DIVA and tubulin, and that the single SNP does not have the function.

### (3) Tubulin and Osteoarthritis

Tubulins and microtubules have essential roles in protein trafficking and secretion. Microtubules are also reported to regulate chondrocyte differentiation. The addition of colchicine, an agent that depolymerizes microtubules, results in lower amount of collagen and glycosaminoglycan in chondrocyte (Farquharson, C., D. Lester, et al. (1999). Bobe 25 (4): 405-12). In cartilage of rat osteoarthritis-induced model, a significant reduction of tubulin is observed (Capin-Gutierrez, N., P. Talamas-Rohana, et al. (2004). Histol Histopathol 19(4): 1125-32.). These reports suggest that tubulins and microtubules might be involved in osteoarthritis pathogenesis. It is speculated that DIVA affects osteoarthritis susceptibility by modulating chondrogenic function of tubulin.

TABLE 1
Association of rs3773472 with knee osteoarthritis (KOA) in the genome-wide analysis

| | KOA | | | | | Control | | | | | P value for |
|---|---|---|---|---|---|---|---|---|---|---|---|
| | Genotype | | | | Allele G | Genotype | | | | Allele G | allele |
| Population | CC | CG | GG | Sum | frequency | CC | CG | GG | Sum | frequency | frequency |
| Set A | 52 | 37 | 5 | 94 | 0.250 | 259 | 279 | 77 | 615 | 0352 | 0.0059 |
| Set B | 324 | 272 | 50 | 646 | 0.288 | 240 | 314 | 74 | 628 | 0.368 | 0.000017 |
| Set A + B combined | 376 | 309 | 55 | 740 | 0.283 | 499 | 593 | 151 | 1243 | 0.360 | 0.00000065 |

TABLE 2
Association of the selected tag SNPs with knee osteoarthritis (KOA)

| | KOA | | | | | Control | | | | | | |
|---|---|---|---|---|---|---|---|---|---|---|---|---|
| | Genotype | | | | Allele 2 | Genotype | | | | Allele 2 | Test for allele frequency | |
| dbSNP ID | 11 | 12 | 22 | Sum | frequency | 11 | 12 | 22 | Sum | frequency | P value | Odds ratio (95% CI)* |
| rs618762 | 515 | 101 | 8 | 624 | 0.094 | 536 | 78 | 7 | 621 | 0.074 | 0.077 | 0.77 (0.58-1.03) |
| rs7639618 | 253 | 293 | 95 | 641 | 0.377 | 162 | 327 | 140 | 629 | 0.483 | 0.000000073 | 1.54 (1.32-1.81) |
| rs353093 | 169 | 316 | 142 | 627 | 0.478 | 117 | 317 | 190 | 624 | 0.558 | 0.000062 | 1.38 (1.18-1.61) |
| rs826428 | 457 | 166 | 15 | 638 | 0.154 | 483 | 129 | 16 | 628 | 0.128 | 0.066 | 0.81 (0.65-1.01) |
| rs3773475 | 273 | 302 | 69 | 644 | 0.342 | 196 | 328 | 100 | 624 | 0.423 | 0.000024 | 1.41 (1.20-1.66) |
| rs11713836 | 317 | 266 | 49 | 632 | 0.288 | 255 | 291 | 77 | 623 | 0.357 | 0.00021 | 1.37 (1.16-1.63) |
| rs1318937 | 253 | 301 | 77 | 631 | 0.361 | 206 | 311 | 104 | 621 | 0.418 | 0.0033 | 1.27 (1.08-1.50) |
| rs3732728 | 349 | 251 | 43 | 643 | 0.262 | 287 | 283 | 59 | 629 | 0.319 | 0.0016 | 1.32 (1.11-1.56) |
| rs2291853 | 192 | 318 | 135 | 645 | 0.456 | 148 | 325 | 156 | 629 | 0.506 | 0.011 | 1.22 (1.05-1.43) |
| rs3773472 | 324 | 272 | 50 | 646 | 0.288 | 240 | 314 | 74 | 628 | 0.368 | 0.000017 | 1.44 (1.22-1.70) |
| rs3773469 | 228 | 315 | 102 | 645 | 0.402 | 163 | 336 | 126 | 625 | 0.470 | 0.00054 | 1.32 (1.13-1.54) |
| rs1287467 | 479 | 144 | 16 | 639 | 0.138 | 475 | 148 | 6 | 629 | 0.127 | 0.43 | 0.91 (0.73-1.15) |

CI: confidence interval. The population set B was genotyped.
Allele 1 and allele 2 indicate the major and minor allele in the KOA population, respectively.
11, 12 and 22 indicate homozygote of allele 1, heterozygote and homozygote of allele 2, respectively.
*Odds ratio for allele 1 vs. allele 2.

TABLE 3
Clinical parameters and the genotype of rs7639618

| | | Genotype | | | | | | |
|---|---|---|---|---|---|---|---|---|
| | Popu- | GG | | GA | | AA | | |
| Variable | lation | Mean | SD | Mean | SD | Mean | SD | P value |
| Age | Set B | 71.9 | 7.0 | 71.7 | 8.3 | 72.7 | 7.7 | 0.42 |
| | KOA | | | | | | | |
| | Set B | 54.9 | 15.0 | 53.9 | 15.7 | 53.8 | 15.7 | 0.79 |
| | Control | | | | | | | |
| BMI | Set B | 24.8 | 3.2 | 24.7 | 3.5 | 25.4 | 4.0 | 0.31 |
| | KOA | | | | | | | |
| | Set B | 25.9 | 3.5 | 25.1 | 3.2 | 25.3 | 3.4 | 0.35 |
| | Control | | | | | | | |
| Sex | Set B | 18.6 | | 16.0 | | 18.9 | | 0.68 |
| | KOA | | | | | | | |
| (Male %) | Set B | 48.1 | | 58.4 | | 51.4 | | 0.08 |
| | Control | | | | | | | |

BMI: body mass index.
The P values between ages and BMIs were calculated using Kruskal-Wallis test.
The P value between sex distributions was calculated using χ² test.

TABLE 4
Assessment of the population stratification

| | Significance | Estimate of | | | Threshold of | | |
|---|---|---|---|---|---|---|---|
| | of | inflation factor | Highest association | | significance | | Highest/ |
| Population | stratification | (95% upper | | Chi-square | | Chi-square | threshold |
| set | (P value) | bound) | P value | statistic | P value | statistic | (chi-square) |
| Set B | 0.943 | 1 (<1.604) | 0.000000073 | 28.99 | 0.000023 | 17.91 | 1.618 |

We genotyped 25 unlinked SNPs for our case-control population. We assessed stratification by calculating significance of stratification using the method of Pritchard and Rosenberg¹, and by estimating the magnitude of stratification using the method of genomic control². We set a threshold of P value as 0.05/2164 (the number of analyzed polymorphisms using the population set 2). The 95th percentile upper bounds of inflation factors (1.604) were lower than highest chi-square statistic/threshold chi-square statistic (1.618), indicating the population stratification is unlikely as an explanation for the positive association.
References
¹Pritchard, J. K. & Rosenberg, N. A. Use of unlinked genetic markers to detect population stratification in association studies. Am J Hum Genet 65, 220-8 (1999).
²Freedman, M. L. et al. Assessing the impact of population stratification on genetic association studies. Nat Genet 36, 388-93 (2004).

TABLE 5
Replication of association of rs7639618 with knee osteoarthritis (KOA)

| | KOA | | | | | Control | | | | | Test for allele frequency | |
|---|---|---|---|---|---|---|---|---|---|---|---|---|
| | Genotype | | | | Allele A | Genotype | | | | Allele A | P | Odds ratio |
| Population | GG | GA | AA | Sum | frequency | GG | GA | AA | Sum | frequency | value | (95% CI) |
| Set C | 99 | 107 | 36 | 242 | 0.370 | 166 | 222 | 95 | 483 | 0.427 | 0.038 | 1.27 (1.01-1.59) |

CI: confidence interval.

TABLE 6
Association of rs7639618 with hip osteoarthritis (HOA)
after stratification by presence of acetabular dysplasia

| | HOA | | | | | | |
|---|---|---|---|---|---|---|---|
| | Genotype | | | | Allele A | Test for allele frequency* | |
| Case population | GG | GA | AA | Sum | frequency | P value | Odds ratio (95% CI) |
| HOA all | 244 | 396 | 160 | 800 | 0.448 | 0.062 | 1.15 (0.99-1.33) |
| HOA AD (+) | 158 | 264 | 112 | 534 | 0.457 | 0.22 | 1.11 (0.94-1.31) |
| HOA AD (−) | 86 | 132 | 48 | 266 | 0.429 | 0.037 | 1.24 (1.01-1.52) |

HOA: hip osteoarthritis,
AD: acetabular dysplasia of the hip,
CI: confidence interval.
*Set B control was used as the control population.

TABLE 7
List of SNPs in and around DIVA

| | Chromosomal | | |
|---|---|---|---|
| | position | | |
| dbSNP ID | (Chromosome 3) | Position in DIVA | r² value* |
| rs1287464 | 15222800 | Promotor | 0.62 |
| | | (CAPN7 exon 1) | |
| rs826428 | 15213896 | Intron | 0.05 |
| rs9864422 | 15202256 | Intron | 0.77 |
| rs9853971 | 15200351 | Intron | 0.91 |
| rs353093 | 15199987 | Intron | 0.73 |
| rs9849394 | 15199646 | Intron | 0.95 |
| rs735659 | 15198998 | Intron | 0.95 |
| rs2129947 | 15197266 | Intron | 0.90 |
| rs9833546 | 15196936 | Intron | 0.91 |
| rs9310470 | 15194872 | Intron | 0.95 |
| rs11718863 | 15191707 | Tyr169Asn | 0.95 |
| rs7639618 | 15191433 | Cys260Tyr | 1.00 |
| rs7637255 | 15191006 | Intron | 1.00 |
| rs7637184 | 15190926 | Intron | 1.00 |
| rs28713191 | 15190298 | Intron | 0.95 |
| rs4685241 | 15190012 | Intron | 1.00 |
| rs618762 | 15189247 | Intron | 0.04 |
| rs6781213 | 15187276 | Intron | 0.95 |
| rs28636560 | 15187045 | 3′-UTR | 0.91 |
| rs7636348 | 15186971 | 3′-UTR | 1.00 |
| rs9827530 | 15186398 | Intron | 1.00 |
| rs10460974 | 15184402 | Intron | 0.95 |
| rs11705926 | 15181721 | 3′-UTR | 1.00 |
| rs1287456 | 15180930 | 3′-flanking region | 0.73 |
| rs4685239 | 15177890 | 3′-flanking region | 1.00 |

*Calculated between rs7639618 (the most associated tag SNP) and each SNP.

TABLE 8
Association of the additional tag SNPs in DIVA region with knee osteoarthritis

| | Case | | | | | Control | | | | | Test for allele frequency | |
|---|---|---|---|---|---|---|---|---|---|---|---|---|
| | Genotype | | | | Allele 2 | Genotype | | | | Allele 2 | | Odds ratio (95% |
| dbSNP ID | 11 | 12 | 22 | Sum | frequency | 11 | 12 | 22 | Sum | frequency | P value | CI)* |
| rs1287464 | 166 | 320 | 153 | 639 | 0.490 | 113 | 311 | 199 | 623 | 0.569 | 0.000067 | 1.38 (1.18-1.61) |
| rs9864422 | 292 | 281 | 65 | 638 | 0.322 | 194 | 325 | 106 | 625 | 0.430 | 0.000000024 | 1.59 (1.35-1.86) |
| rs11718863 | 255 | 294 | 95 | 644 | 0.376 | 162 | 327 | 137 | 626 | 0.480 | 0.00000011 | 1.53 (1.31-1.80) |

CI: confidence interval. The population set B was genotyped.
Allele 1 and allele 2 indicate the major and minor allele in the KOA population, respectively.
11, 12 and 22 indicate homozygote of allele 1, heterozygote and homozygote of allele 2, respectively.
*Odds ratio for allele 1 vs. allele 2.

TABLE 9
Haplotype association analysis

| | | Frequency | | |
|---|---|---|---|---|
| Tag SNP set | Haplotype | Case | Control | P value |
| 12 SNPs in LD block | I | 0.405 | 0.347 | 0.0030 |
| | II | 0.216 | 0.277 | 0.00051 |
| | III | 0.064 | 0.047 | 0.065 |
| | IV | 0.061 | 0.049 | 0.20 |
| | V | 0.035 | 0.029 | 0.42 |
| | VI | 0.026 | 0.024 | 0.86 |
| | VII | 0.024 | 0.026 | 0.76 |
| | VIII | 0.023 | 0.021 | 0.78 |
| | IX | 0.022 | 0.018 | 0.44 |
| | X | 0.021 | 0.029 | 0.22 |
| | XI | 0.021 | 0.019 | 0.75 |
| | XII | 0.013 | 0.011 | 0.61 |
| | XIII | 0.012 | 0.033 | 0.00037 |
| | XIV | 0.010 | 0.005 | 0.16 |
| 7 SNPs in DIVA region | I | 0.507 | 0.432 | 0.00020 |
| | II | 0.319 | 0.424 | 0.000000096 |
| | III | 0.091 | 0.074 | 0.13 |
| | IV | 0.059 | 0.053 | 0.53 |
| | V | 0.016 | 0.010 | 0.22 |

All haplotypes with frequency of >1% in the KOA population of set B are shown.

TABLE 10
Haplotype frequencies of rs11718863 and rs7639618

| | | rs7639618 | | Haplotype | | |
|---|---|---|---|---|---|---|
| rs11718863 | | | AA | frequency | | |
| Allele | AA at 169 | Allele | at 260 | Case | Control | P value |
| T | Y | G | C | 0.62 | 0.52 | 0.00000012 |
| A | N | A | Y | 0.38 | 0.48 | 0.00000011 |
| A | N | G | C | 0.0023 | 0.0016 | 0.67 |
| T | Y | A | Y | 0 | 0 | — |

AA: Amino acid.
Haplotype frequencies were calculated using set B.

TABLE 11
Clinical parameters of the populations

| | | | | Age | | | |
|---|---|---|---|---|---|---|---|
| | Number of | Male | Female | (years) | | BMI | |
| Population | individuals | (%) | (%) | Mean | SD | Mean | SD |
| Set A KOA | 94 | 9.6 | 90.4 | 72.1 | 7.3 | 25.7 | 4.0 |
| Set A Control | 658 | 55.5 | 44.5 | 48.5 | 21.6 | 27.3 | 6.9 |
| Set B KOA | 646 | 18.0 | 82.0 | 72.0 | 7.7 | 24.8 | 3.5 |
| Set B Control | 631 | 54.2 | 45.8 | 54.1 | 15.5 | 25.3 | 3.3 |
| Set C KOA | 242 | 24.8 | 75.2 | 71.4 | 7.7 | 24.4 | 3.3 |
| Set C Control | 485 | 36.7 | 63.3 | 68.2 | 8.7 | 22.5 | 2.8 |
| HOA | 803 | 7.5 | 92.5 | 57.8 | 10.4 | 22.7 | 3.1 |

“OA” and “HOA” show knee osteoarthritis and hip osteoarthritis.
MI: body mass index.

### INDUSTRIAL APPLICABILITY

According to the present invention, it becomes possible to diagnose susceptibility to osteoarthritis by examining (by genetic diagnosis, hemodiagnosis, etc.) the disease susceptibility polymorphism of a DIVA gene.

### BRIEF DESCRIPTION OF DRAWINGS

FIG. 1 shows the nucleotide and deduced amino acid sequences of the human DIVA gene. The two domains that were homologous with von Willebrand factor domain A are underlined. The stop codon is indicated by an asterisk and the polyA signal is in a open box.

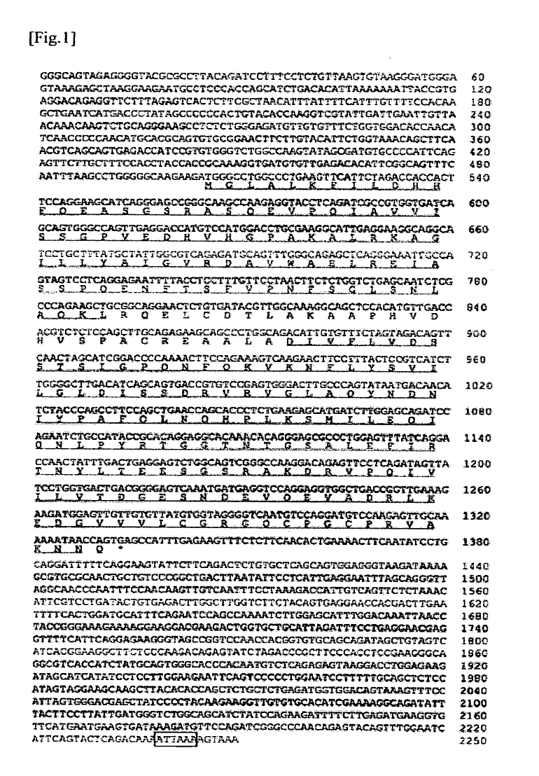

FIG. 2 shows the transcriptional activity of the DIVA promoter in HEK293 (a) and HCS-2/8 (b).

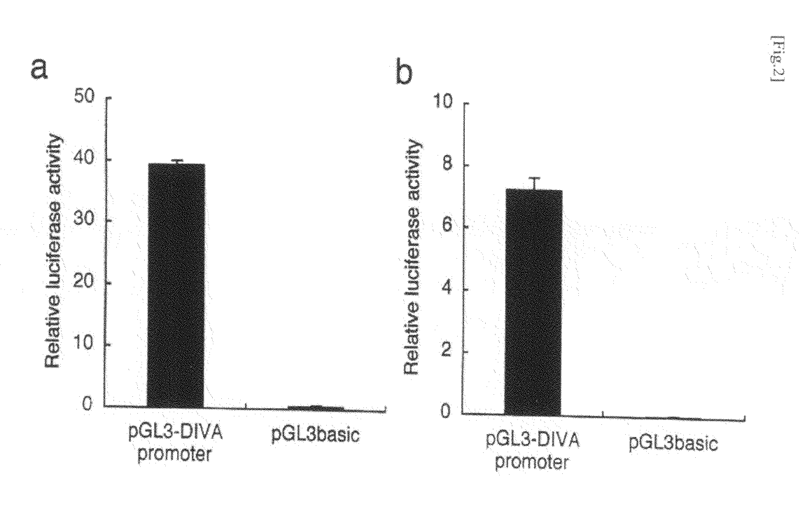

FIG. 3 shows the DIVA expression. (a) shows northern blotting in human cartilage cells. (b) shows DIVA expression in various human tissues. DIVA is specifically expressed in cartilage. Data represent the mean ratios of DIVA mRNA to glyceraldehyde-3-phosphate dehydrogenase (GAP DH) mRNA±standard error.

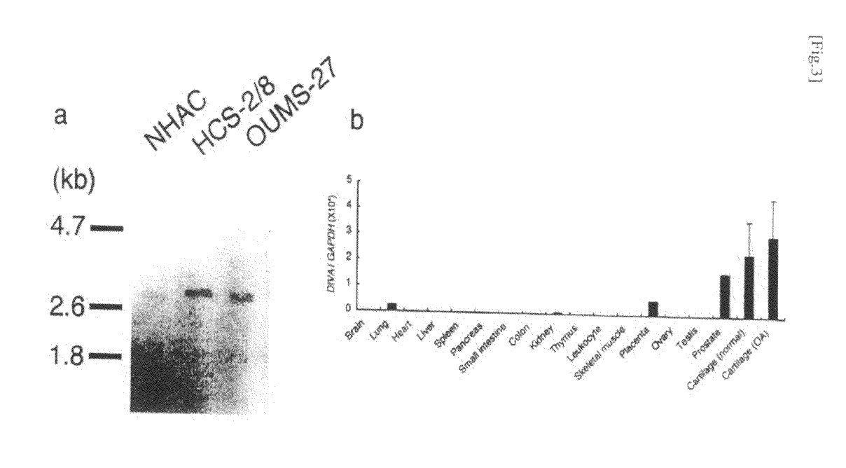

FIG. 4 shows the binding of DIVA with tubulin. (a) shows the results obtained by subjecting a sample to immunoprecipitation with S-protein, then performing SDS-PAGE, and then subjecting the resultant to silver impregnation for detection. The arrow shows the position of a β-tubulin band that has been confirmed by MALDI/TOF mass spectrometry. (b) shows the results obtained by confirming the binding of DIVA to tubulin by the Western blot method. (c) shows the results obtained by confirming by Coomassie staining that a genetically modified DIVA protein having an estimated size has been expressed, after completion of SDS-PAGE. The term “Y-C” means that the amino acid at position 169 is Tyr and the amino acid at position 260 is Cys. (d) shows the results obtained by measuring the binding force of a DIVA isoform by a solid-phase binding experiment. The well of a microplate was coated with a genetically modified DIVA protein or a control protein, and it was then incubated in the presence or absence of bovine tubulin. The data is given in the form of a mean value from 2 times of assays ±a standard error. The experiment was repeated 3 times. As a result, the same results were obtained. *P<0.01 (Student's T-test).

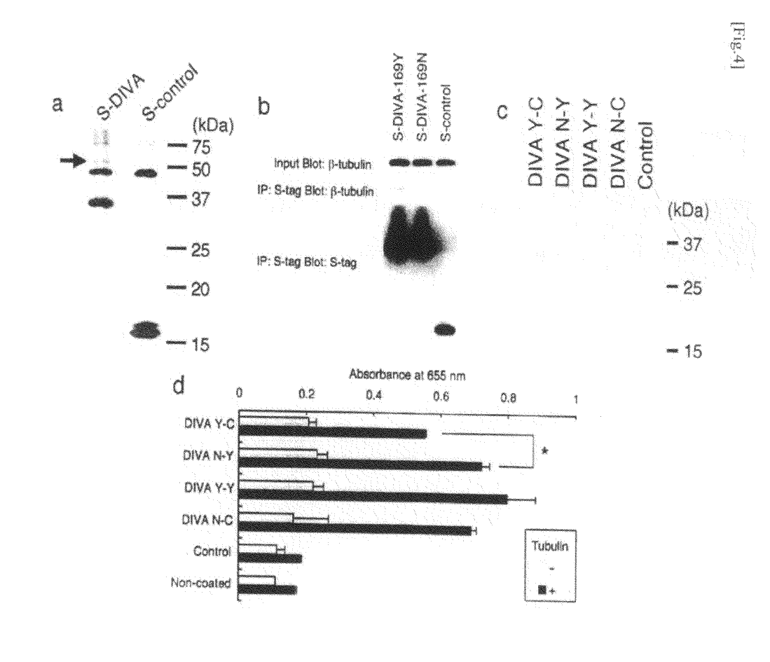

FIG. 5 shows the results obtained by quantifying the expression of DIVA, type-II collagen, and aggrecan genes by a real-time PCR method, using DIVA-specific siRNA.

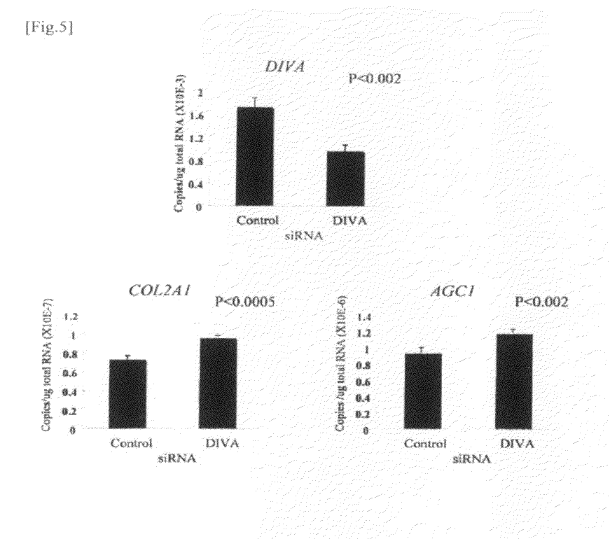

FIG. 6 shows the results obtained by forcing OUMS-27 cells to express a DIVA gene and then analyzing the action of the DIVA gene on the expression of type-II collagen (COL2A1) and aggrecan (AGC1).

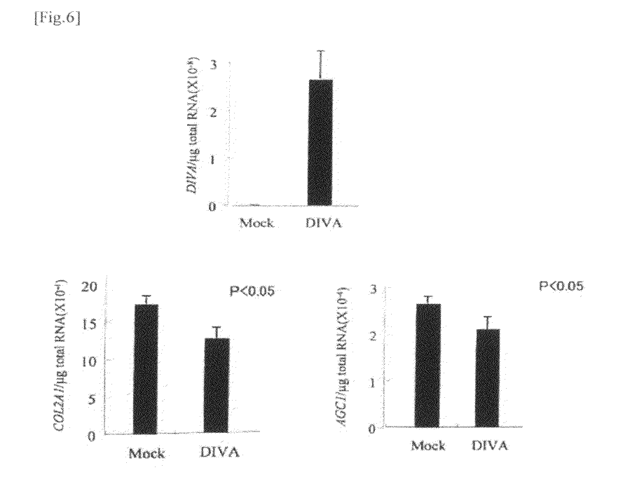

FIG. 7 shows the results of Western blotting using a DIVA antibody.

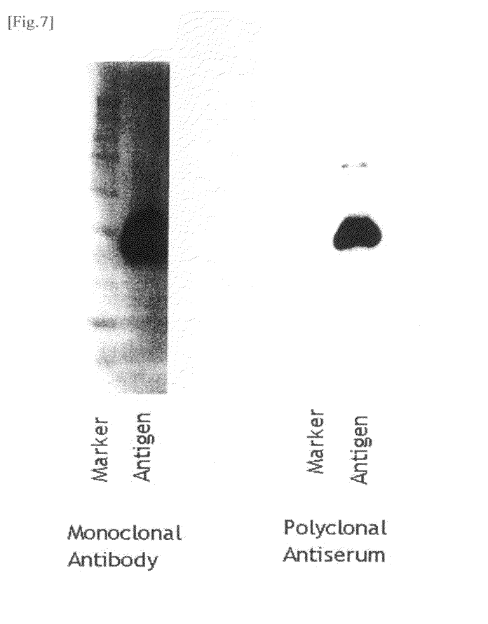

1. A method for diagnosing a genetic susceptibility of a subject to osteoarthritis, which comprises detecting at least one polymorphism selected from polymorphisms existing in a gene (Dual Intracellular on Willebrand factor A gene; DIVA gene), encoding the amino acid sequence of SEQ ID NO: 2 or an amino acid sequence substantially homologous thereto, in a DNA-containing sample collected from the subject, wherein an allele frequency of one of alleles is higher in arbitrary osteoarthritis group than in arbitrary non-osteoarthritis group.

2. The method according to claim 1, wherein the polymorphism is selected from the group consisting of the polymorphisms of registration numbers rs9864422, rs7639618, and rs11718863 in the NCBI SNP Database, and polymorphisms that are in a linkage disequilibrium state with the polymorphisms, having a linkage disequilibrium coefficient D′ of 0.9 or greater.

3. The method according to claim 2, wherein the subject is determined to have high genetic susceptibility to osteoarthritis when a genotype of registration number rs11718863 in the NCBI SNP Database is T and a genotype of registration number rs7639618 is G.

4. The method according to claim 1, wherein the osteoarthritis is knee osteoarthritis.

5. The method according to claim 1, wherein presence or absence of a genetic polymorphism is detected, the genetic polymorphism causing an amino acid at position 169 in the amino acid sequence of SEQ ID NO: 2 to alter from Asn to an amino acid other than Asn.

6. The method according to claim 1, wherein presence or absence of a genetic polymorphism is detected, the genetic polymorphism causing an amino acid at position 260 in the amino acid sequence of SEQ ID NO: 2 to alter from Tyr to an amino acid other than Tyr.

7. A method for screening an agent for preventing and/or treating osteoarthritis, which comprises administering a test substance to cells that express a gene encoding a protein consisting of the amino acid sequence of SEQ ID NO: 2 or an amino acid sequence substantially homologous thereto, and selecting a substance that inhibits expression or function of the protein.

8. An agent for preventing and/or treating osteoarthritis, which comprises an agent for inhibiting expression or function of a protein consisting of the amino acid sequence of SEQ ID NO: 2 or an amino acid sequence substantially homologous thereto.

9. A protein consisting of the following amino acid sequence (a) or (b):
(a) the amino acid sequence of SEQ ID NO: 2; or
(b) an amino acid sequence, which comprises a deletion, substitution, insertion, and/or addition of one or more amino acid residues with respect to the amino acid sequence of SEQ ID NO: 2, and which is able to bind to tubulin.

10. DNA encoding the protein of claim 9.

11. DNA consisting of any one of the following nucleotide sequences (a) to (c):
(a) the nucleotide sequence of SEQ ID NO: 1;
(b) a nucleotide sequence, which comprises a deletion, substitution, insertion, and/or addition of one or more nucleotides with respect to the nucleotide sequence of SEQ ID NO: 1, and which encodes an amino acid sequence that is able to bind to tubulin; and
(c) a nucleotide sequence, which hybridizes with the nucleotide sequence of SEQ ID NO: 1 or a sequence complementary thereto under stringent conditions, and which encodes an amino acid sequence that is able to bind to tubulin.
